SEQUENCE LISTING (53 sequences)

SEQ ID NO: 1 - protein, 124 aa, Saccharomyces cerevisiae
EHVAFGSEDIENTLAKMDDGQLDGLAFGAIQLDGDGNILQYNAAEGDITGRDPKQVIGKNFFKDVAPGTDSPEFYGKFKEGVASGNLNTMFEWMIPTSRGPTKVKVHMKKALSGDSYWVFVKRV

SEQ ID NO: 2 - DNA, 372 bp, Saccharomyces cerevisiae
  source 1..372: mol_type = genomic DNA
gaacatgtggcgtttggaagtgaggacattgagaatacgcttgcgaagatggatgatggtcaactggatggtcttgcctttggagcaattcagttggatggcgatggtaacatcttgcagtacaatgccgccgagggtgatattacaggacgtgatcccaaacaagtgattggaaaaaattttttcaaagatgtagcgcctggcactgactcacccgagttttacggtaagttcaaagaaggcgtggcttccggtaatcttaatacgatgtttgagtggatgatccccactagccgtggacccaccaaggtgaaagtgcatatgaagaaggccttatcgggcgatagctactgggtgttcgttaaacgtgtt

SEQ ID NO: 3 - protein, 124 aa, synthetic construct
  REGION 1..124: note = Synthetic polypeptide
EHVAFGSEDIENTLAKMDDGQLDGLAFGAIQLDGDGNILQYNAAEGDITGRDPKQVIGKNLFKDVACGTRSSEFYGKFKEGVASGNLNTMFEWMIPTSRGPTKVKVHMKKALSGDSYWVFVKRV

SEQ ID NO: 4 - DNA, 372 bp, synthetic construct
  misc_feature 1..372: note = Synthetic polynucleotide
  source 1..372: mol_type = other DNA
gaacatgtggcgtttggaagtgaggacattgagaatacgcttgcgaagatggatgatggtcaactggatggtcttgcctttggagcaattcagttggatggcgatggtaacatcttgcagtacaatgccgccgagggtgatattacaggacgtgatcccaaacaagtgattggaaaaaatctgttcaaagatgtagcgtgcggcactcgttcaagcgagttttacggtaagttcaaagaaggcgtggcttccggtaatcttaatacgatgtttgagtggatgatccccactagccgtggacccaccaaggtgaaagtgcatatgaagaaggccttatcgggcgatagctactgggtgttcgttaaacgtgtt

SEQ ID NO: 5 - protein, 237 aa, synthetic construct
  REGION 1..237: note = Synthetic polypeptide
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 6 - DNA, 711 bp, synthetic construct
  misc_feature 1..711: note = Synthetic polynucleotide
  source 1..711: mol_type = other DNA
atggtgtctaaaggagaggaacttatcaaagaaaatatgcacatgaagctttacatggaaggaacagtggacaatcaccattttaaatgtacatcagaaggcgagggtaaaccttatgaggggacgcaaaccatgcgtatcaaggtcgtagagggcggccctttgcctttcgctttcgacattcttgcaacctcattcttgtatggctccaagacttttatcaaccatacacaaggcattcccgatttctttaaacaatcgttccctgaaggttttacatgggaacgtgtaacaacatatgaagatgggggagttttaactgccacacaggatacatctttacaggatggctgcctgatctataatgtaaagatccgtggagtgaactttacctcgaacggccccgtcatgcagaaaaagacccttgggtgggaggcctttacggaaacgctttaccccgcggacggaggtctggaaggacgtaatgacatggcgctgaagcttgtcggaggatcccatctgatcgcaaatgctaagaccacctatcgtagcaagaaacctgctaaaaacttaaaaatgcctggtgtttactacgtggactatcgtcttgagcgtattaaggaggcaaataacgaaacctatgttgaacaacacgaggtcgctgtggcccgctattgcgacttgccctcgaagctggggcataagttgaat

SEQ ID NO: 7 - protein, 236 aa, synthetic construct
  REGION 1..236: note = Synthetic polypeptide
VSKGEETIMSVIKPDMKIKLRMEGNVNGHAFVIEGEGSGKPFEGIQTIDLEVKEGAPLPFAYDILTTAFHYGNRVFTKYPRKIPDYFKQSFPEGYSWERSMTYEDGGICNATNDITMEEDSFINKIHFKGTNFPPNGPVMQKRTVGWEVSTEKMYVRDGVLKGDVKMKLLLKGGSHYRCDFRTTYKVKQKAVKLPKAHFVDHRIEILSHDKDYNKVKLYEHAVARNSTDSMDELYK

SEQ ID NO: 8 - DNA, 708 bp, synthetic construct
  misc_feature 1..708: note = Synthetic polynucleotide
  source 1..708: mol_type = other DNA
gttagcaagggcgaggagaccatcatgagcgtgatcaagccggacatgaagatcaagctgcgcatggagggcaacgtgaacggccatgcctttgtgatcgagggcgagggcagcggtaagccgtttgagggcatccagaccatcgacctggaggttaaggaaggcgcaccgctgccgtttgcctacgacatcctgaccaccgcattccactacggcaaccgcgtgttcaccaagtacccgcgcaagatcccggactacttcaagcagagcttcccggagggctacagttgggaacgcagcatgacctacgaggacggcggtatctgcaacgccaccaacgacatcaccatggaagaagatagcttcatcaacaagatccacttcaagggcacaaacttcccgccgaatggtccggttatgcagaagcgcaccgttggctgggaggtgagcaccgagaagatgtatgtgcgcgacggtgtgctgaagggcgacgtgaagatgaagctgctgctgaagggtggcagccactaccgctgcgacttccgcaccacctacaaagttaagcaaaaggcagtgaagttaccgaaggcccacttcgtggaccaccgcatcgaaatcctgagccacgacaaggactataacaaagtgaagctgtacgagcacgccgtggcccgtaacagcaccgacagcatggatgagctgtacaaa

SEQ ID NO: 9 - DNA, 20 bp, synthetic construct
  misc_feature 1..20: note = Synthetic polynucleotide
  source 1..20: mol_type = other DNA
atcctccttcaatacatccc

SEQ ID NO: 10 - DNA, 20 bp, synthetic construct
  misc_feature 1..20: note = Synthetic polynucleotide
  source 1..20: mol_type = other DNA
tatctcatcaatcccacact

SEQ ID NO: 11 - DNA, 20 bp, synthetic construct
  misc_feature 1..20: note = Synthetic polynucleotide
  source 1..20: mol_type = other DNA
acactaccaccatttcctat

SEQ ID NO: 12 - DNA, 20 bp, synthetic construct
  misc_feature 1..20: note = Synthetic polynucleotide
  source 1..20: mol_type = other DNA
aaacacacactaaaccaccc

SEQ ID NO: 13 - DNA, 20 bp, synthetic construct
  misc_feature 1..20: note = Synthetic polynucleotide
  source 1..20: mol_type = other DNA
actccactactactcactct

SEQ ID NO: 14 - DNA, 20 bp, synthetic construct
  misc_feature 1..20: note = Synthetic polynucleotide
  source 1..20: mol_type = other DNA
aactcatctcaatcctccca

SEQ ID NO: 15 - DNA, 20 bp, synthetic construct
  misc_feature 1..20: note = Synthetic polynucleotide
  source 1..20: mol_type = other DNA
accctctaacttccatcaca

SEQ ID NO: 16 - DNA, 20 bp, synthetic construct
  misc_feature 1..20: note = Synthetic polynucleotide
  source 1..20: mol_type = other DNA
aatactctcccacctcaact

SEQ ID NO: 17 - DNA, 20 bp, synthetic construct
  misc_feature 1..20: note = Synthetic polynucleotide
  source 1..20: mol_type = other DNA
accacaacccattcctttca

SEQ ID NO: 18 - DNA, 20 bp, synthetic construct
  misc_feature 1..20: note = Synthetic polynucleotide
  source 1..20: mol_type = other DNA
tctatcatctccaaaccaca

SEQ ID NO: 19 - DNA, 20 bp, synthetic construct
  misc_feature 1..20: note = Synthetic polynucleotide
  source 1..20: mol_type = other DNA
tttctaccactaatcaaccc

SEQ ID NO: 20 - DNA, 20 bp, synthetic construct
  misc_feature 1..20: note = Synthetic polynucleotide
  source 1..20: mol_type = other DNA
tccaactcatctctaatctc

SEQ ID NO: 21 - DNA, 20 bp, synthetic construct
  misc_feature 1..20: note = Synthetic polynucleotide
  source 1..20: mol_type = other DNA
accctttacaaacacaccct

SEQ ID NO: 22 - DNA, 20 bp, synthetic construct
  misc_feature 1..20: note = Synthetic polynucleotide
  source 1..20: mol_type = other DNA
ttcctaacaaatcacatccc

SEQ ID NO: 23 - DNA, 20 bp, synthetic construct
  misc_feature 1..20: note = Synthetic polynucleotide
  source 1..20: mol_type = other DNA
tcctattctcaacctaacct

SEQ ID NO: 24 - DNA, 20 bp, synthetic construct
  misc_feature 1..20: note = Synthetic polynucleotide
  source 1..20: mol_type = other DNA
ataaatcattcccactaccc

SEQ ID NO: 25 - DNA, 20 bp, synthetic construct
  misc_feature 1..20: note = Synthetic polynucleotide
  source 1..20: mol_type = other DNA
tatccttcaatccctccaca

SEQ ID NO: 26 - DNA, 20 bp, synthetic construct
  misc_feature 1..20: note = Synthetic polynucleotide
  source 1..20: mol_type = other DNA
acccaacactcataacatcc

SEQ ID NO: 27 - DNA, 20 bp, synthetic construct
  misc_feature 1..20: note = Synthetic polynucleotide
  source 1..20: mol_type = other DNA
acattacacctcattctccc

SEQ ID NO: 28 - DNA, 20 bp, synthetic construct
  misc_feature 1..20: note = Synthetic polynucleotide
  source 1..20: mol_type = other DNA
tactacaaacccataatccc

SEQ ID NO: 29 - DNA, 20 bp, synthetic construct
  misc_feature 1..20: note = Synthetic polynucleotide
  source 1..20: mol_type = other DNA
tttactccctacacctccaa

SEQ ID NO: 30 - DNA, 20 bp, synthetic construct
  misc_feature 1..20: note = Synthetic polynucleotide
  source 1..20: mol_type = other DNA
actttccacatactatccca

SEQ ID NO: 31 - DNA, 20 bp, synthetic construct
  misc_feature 1..20: note = Synthetic polynucleotide
  source 1..20: mol_type = other DNA
ttctccctctatcaactcta

SEQ ID NO: 32 - DNA, 20 bp, synthetic construct
  misc_feature 1..20: note = Synthetic polynucleotide
  source 1..20: mol_type = other DNA
ttcttccctcaatcttcatc

SEQ ID NO: 33 - DNA, 20 bp, synthetic construct
  misc_feature 1..20: note = Synthetic polynucleotide
  source 1..20: mol_type = other DNA
acccttactactacatcatc

SEQ ID NO: 34 - DNA, 20 bp, synthetic construct
  misc_feature 1..20: note = Synthetic polynucleotide
  source 1..20: mol_type = other DNA
aatctcaccttccacttcac

SEQ ID NO: 35 - DNA, 20 bp, synthetic construct
  misc_feature 1..20: note = Synthetic polynucleotide
  source 1..20: mol_type = other DNA
tcctaacaaccaactactcc

SEQ ID NO: 36 - DNA, 20 bp, synthetic construct
  misc_feature 1..20: note = Synthetic polynucleotide
  source 1..20: mol_type = other DNA
acctttctccatacccaact

SEQ ID NO: 37 - DNA, 20 bp, synthetic construct
  misc_feature 1..20: note = Synthetic polynucleotide
  source 1..20: mol_type = other DNA
tctatcattaccctcctcct

SEQ ID NO: 38 - DNA, 20 bp, synthetic construct
  misc_feature 1..20: note = Synthetic polynucleotide
  source 1..20: mol_type = other DNA
tcctcatcttactccctcta

SEQ ID NO: 39 - DNA, 20 bp, synthetic construct
  misc_feature 1..20: note = Synthetic polynucleotide
  source 1..20: mol_type = other DNA
tattcaccttacaaaccctc

SEQ ID NO: 40 - DNA, 20 bp, synthetic construct
  misc_feature 1..20: note = Synthetic polynucleotide
  source 1..20: mol_type = other DNA
tcaaactttccaaccacctc

SEQ ID NO: 41 - DNA, 20 bp, synthetic construct
  misc_feature 1..20: note = Synthetic polynucleotide
  source 1..20: mol_type = other DNA
ttacctctaaccctccattc

SEQ ID NO: 42 - DNA, 20 bp, synthetic construct
  misc_feature 1..20: note = Synthetic polynucleotide
  source 1..20: mol_type = other DNA
acaccatttatccactcctc

SEQ ID NO: 43 - DNA, 20 bp, synthetic construct
  misc_feature 1..20: note = Synthetic polynucleotide
  source 1..20: mol_type = other DNA
tcccaactaacctaacattc

SEQ ID NO: 44 - DNA, 20 bp, synthetic construct
  misc_feature 1..20: note = Synthetic polynucleotide
  source 1..20: mol_type = other DNA
acatcctaactacaaccttc

SEQ ID NO: 45 - DNA, 20 bp, synthetic construct
  misc_feature 1..20: note = Synthetic polynucleotide
  source 1..20: mol_type = other DNA
atcctcactacatcatccac

SEQ ID NO: 46 - DNA, 20 bp, synthetic construct
  misc_feature 1..20: note = Synthetic polynucleotide
  source 1..20: mol_type = other DNA
tctcacaccactttcctcat

SEQ ID NO: 47 - DNA, 20 bp, synthetic construct
  misc_feature 1..20: note = Synthetic polynucleotide
  source 1..20: mol_type = other DNA
tccctatcaatctccataac

SEQ ID NO: 48 - DNA, 20 bp, synthetic construct
  misc_feature 1..20: note = Synthetic polynucleotide
  source 1..20: mol_type = other DNA
ttatccatccctcttcctac

SEQ ID NO: 49 - DNA, 20 bp, synthetic construct
  misc_feature 1..20: note = Synthetic polynucleotide
  source 1..20: mol_type = other DNA
tcacctctaactcattacct

SEQ ID NO: 50 - DNA, 20 bp, synthetic construct
  misc_feature 1..20: note = Synthetic polynucleotide
  source 1..20: mol_type = other DNA
tcctacaacatccttcctaa

SEQ ID NO: 51 - DNA, 20 bp, synthetic construct
  misc_feature 1..20: note = Synthetic polynucleotide
  source 1..20: mol_type = other DNA
atctcccttctcttctcata

SEQ ID NO: 52 - DNA, 20 bp, synthetic construct
  misc_feature 1..20: note = Synthetic polynucleotide
  source 1..20: mol_type = other DNA
attacacctcaacccacaca

SEQ ID NO: 53 - DNA, 80 bp, synthetic construct
  misc_feature 1..80: note = Synthetic polynucleotide
  source 1..80: mol_type = other DNA
atcctccttcaatacatcccaaacacacactaaaccacccaactcatctcaatcctcccaatctcccttctcttctcata

(19) United States
(12) Patent Application Publication
(10) Pub. No.: US 2024/0175081 A1
(43) Pub. Date: May 30, 2024

(54) SYSTEMS AND METHODS FOR HIGH-THROUGHPUT IMAGE-BASED SCREENING

(71) Applicant: President and Fellows of Harvard College, Cambridge, MA (US)

(72) Inventors: Xiaowei Zhuang, Cambridge, MA (US); George Alexander Emanuel, Cambridge, MA (US); Jeffrey R. Moffitt, Cambridge, MA (US)

(73) Assignee: President and Fellows of Harvard College, Cambridge, MA (US)

(21) Appl. No.: 18/461,672

(22) Filed: Sep. 6, 2023

Related U.S. Application Data
(63) Continuation of application No. 16/616,833, filed on Nov. 25, 2019, now Pat. No. 11,788,123.
(60) Provisional application No. 62/511,920, filed on May 26, 2017.

(51) Int. Cl. C12Q 1/6841; C40B 30/04; C40B 70/00

(52) U.S. Cl. CPC C12Q 1/6841; C40B 30/04; C40B 70/00; C12Q 2525/161; C12Q 2563/179; C12Q 2565/514

(57) ABSTRACT

The present invention generally relates to imaging cells, for example, to determine phenotypes and/or genotypes in populations of cells. In some aspects, cells may be analyzed, e.g., imaged, to determine their phenotype, and their genotypes may be determined by exposing the cells to nucleic acid probes, e.g., as in smFISH. MERFISH, FISH, in situ hybridization, or other suitable techniques. In some cases, the cells may be exposed to a nucleic acid comprising an identification portion, which may be used to distinguish the cells from each other. In some embodiments, the cells may be exposed to a nucleic acid comprising an expression portion, e.g. a gene, or coding region for a non-translated RNA, etc., that when expressed, produces a protein, RNA, DNA, or the like that may alter the phenotype of the cell or the variable nucleic acid sequence can consist of promoters, gene regulatory elements, transcription factor binding sites, Cas9 guide RNA coding regions, etc. that otherwise alter the phenotype of the cell. In some embodiments, the modifications that contain either the identification portion, the expression portion, or both may be introduced into the genome of a host organism or as exogenous materials, e.g. plasmids. Such changes may involve the addition of synthetic materials, such as synthetic nucleic acids, or modifications, e.g. deletions or mutations, of the genomic material of the host organism. Other aspects are generally directed to compositions or devices for use in such methods, kits for use in such methods, or the like.

### RELATED APPLICATIONS

This application claims the benefit of U.S. Provisional Patent Application Ser. No. 62/511.920, filed May 26, 2017, entitled “High-Throughput, Image-Based Screening of Genetic Variant Libraries,” by Zhuang, et al., incorporated herein by reference in its entirety.

### GOVERNMENT FUNDING

This invention was made with government support under Grant No. 1R01MH113094-01 awarded by National Institutes of Health. The government has certain rights in the invention.

### FIELD

The present invention generally relates to imaging cells, for example, to determine phenotypes and/or genotypes in populations of cells.

### BACKGROUND

High-throughput screening of genetic perturbations is playing an increasingly important role in advancing biology and biotechnology. For example, by observing the effects of a large number of amino acid changes within a selected protein, large-scale screening allows efficient searches for fluorescent proteins better adapted as bioimaging tools or protein and nucleic acid drugs with desired therapeutic properties. It also allows examining how mutations of a protein or gene regulatory element affect cell function or physiology. Since each cell is composed of many genes, high-throughput screening also allows the effects of inhibition or activation of individual genes or combinations of genes to be tested at the genomic scale, which can help deciphering the effects of genes, gene regulatory networks and cell signaling networks on cellular behaviors.

Screening relies on the ability to select based on the phenotype of interest. Many phenotypes require imaging-based assays to measure. While for a single genetic variant, it is straightforward to introduce genetic variation and measure any image-based phenotype, measuring such phenotypes for many genetic variants is more difficult. It is possible to create and characterize each variant in isolation in many well chambers, but, as the number of variants increases, this approach quickly becomes impractical. Creating all desired variants simultaneously as a pooled library is only moderately more complicated than for a single mutant, but this strategy requires the ability to identify which introduced genetic variation results in which genotype. Although cell sorting methods have been adapted to measure fluorescence intensities for each genetic variant by sorting into multiple intensity bins, high-throughput association of genetic variants to more general image-based phenotypes remains difficult. Accordingly, improvements in such techniques are needed.

### SUMMARY

The present invention generally relates to imaging cells, for example, to determine phenotypes and/or genotypes in populations of cells. The subject matter of the present invention involves, in some cases, interrelated products, alternative solutions to a particular problem, and/or a plurality of different uses of one or more systems and/or articles.

In one aspect, the present invention is generally directed to a method. In one set of embodiments, the method comprises introducing, into a plurality of cells, nucleic acids comprising an identification portion and an expression portion; determining phenotype of the cells; and determining genotype of the plurality of cells by sequentially exposing the plurality of cells to nucleic acid probes, and determining binding of target sequences of the nucleic acid probes within the plurality of cells.

The method, in another set of embodiments, includes introducing, into a plurality of cells, a nucleic acid comprising an identification portion and an expression portion; imaging the plurality of cells to determine phenotype of the cells; and determining genotype of the plurality of cells.

According to yet another set of embodiments, the method comprises acquiring an image of a plurality of cells transfected with a plurality of nucleic acids comprising an identification portion and an expression portion, wherein the cells exhibit imagable differences in phenotype due to expression of the expression portion; and acquiring a plurality of images of the plurality of cells, wherein the images of the cells exhibit differences due to differences in the identification portions of the nucleic acids within the cells.

Still another set of embodiments is generally directed to a method comprising acquiring an image of a plurality of cells transfected with a plurality of nucleic acids comprising an identification portion and an expression portion, wherein the cells exhibit imagable differences in phenotype due to expression of the expression portion; and performing in situ hybridization, FISH, multiplexed FISH, smFISH, CASFISH, and/or MERFISH on the cells.

In one set of embodiments, the method includes acquiring an image of a plurality of cells transfected with a plurality of nucleic acids comprising an identification portion and an expression portion, wherein the cells exhibit imagable differences in phenotype due to expression of the expression portion; and determining genotype of the plurality of cells.

In another set of embodiments, the method includes introducing, into a plurality of cells, a nucleic acid comprising an identification portion and an expression portion; and determining phenotype of the plurality of cells by sequentially exposing the plurality of cells to nucleic acid probes, and determining binding of the target sequences of the nucleic acid probes within the plurality of cells.

According to yet another set of embodiments, the method includes creating plurality of nucleic acids comprising an identification portion and an expression portion, wherein when expressed in cells, the identification portion is identifiable by sequentially exposing the cells to nucleic acid probes.

Another aspect of the present invention is generally directed to a composition. In accordance with one set of embodiments, the composition comprises a plurality of nucleic acids comprising an identification portion and an expression portion. In some cases, when expressed in cells, the identification portion is identifiable by sequentially exposing the cells to nucleic acid probes.

In another set of embodiments, the composition comprises a plurality of nucleic acids comprising an identification portion and an expression portion. In certain embodiments, the identification portion comprises N variable portions, N being at least 3, each variable portion being of at least two possibilities. In some cases, each possible combination of variable portions is present within the plurality of nucleic acids.

The present invention, in still another embodiment, is generally directed to a protein. In accordance with one set of embodiments, for example, the protein may comprise a sequence

### EHVAFGSEDIENTLAKMDDGQLDGLAFGAIQLDGDGNILQYNAAEGDITGRDPKQVI GKNLFKDVACGTRSSEFYGKFKEGVASGNLNTMFEWMIPTSRGPTKVKVHMKKAL SGDSYWVFVKRV (SEQ ID NO: 3).

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C illustrate an image-based screening method in accordance with one embodiment of the invention;

FIGS. 2A-2F illustrate the determination of cells containing barcodes of nucleic acids and the determination of the phenotype of those cells, in another embodiment of the invention;

FIG. 3 illustrates the distribution of the ratio of readout 0 intensity to the readout 1 intensity for reading one bit of the barcode, in one embodiment of the invention;

FIGS. 4A-4G illustrate screening of cells, in yet another embodiment of the invention; and

FIG. 5 illustrates the fluorescence decay and recovery of YFAST upon periodic illumination, in another embodiment of the invention;

FIGS. 6A-6F illustrate additional screening of cells, in yet another embodiment of the invention;

FIG. 7 illustrates the structure of a nucleic acid containing bits, in still another embodiment of the invention.

### DETAILED DESCRIPTION

The present invention generally relates to imaging cells, for example, to determine phenotypes and/or genotypes in populations of cells. In some aspects, cells may be analyzed. e.g., imaged, to determine their phenotype, and their genotypes may be determined by exposing the cells to nucleic acid probes, e.g., as in smFISH, MERFISH, FISH, in situ hybridization, or other suitable techniques. In some cases, the cells may be exposed to a nucleic acid comprising an identification portion, which may be used to distinguish the cells from each other. In some embodiments, the cells may be exposed to a nucleic acid comprising an expression portion, e.g. a gene, or coding region for a non-translated RNA, etc., that when expressed, produces a protein, RNA, DNA, or the like that may alter the phenotype of the cell or the variable nucleic acid sequence can consist of promoters, gene regulatory elements, transcription factor binding sites, Cas9 guide RNA coding regions, etc. that otherwise alter the phenotype of the cell. In some embodiments, the modifications that contain either the identification portion, the expression portion, or both may be introduced into the genome of a host organism or as exogenous materials, e.g. plasmids. Such changes may involve the addition of synthetic materials, such as synthetic nucleic acids, or modifications, e.g. deletions or mutations, of the genomic material of the host organism. Other aspects are generally directed to compositions or devices for use in such methods, kits for use in such methods, or the like.

Thus, according to one aspect, the present invention is generally directed to systems and methods for determining the phenotypes and/or genotypes of populations of cells using imaging. In some cases, relatively large numbers of cells may be studied, e.g., using suitable imaging techniques such as those described herein, to determine their phenotypes and genotypes. In some embodiments, due to the use of such imaging techniques, relatively large number of cells may be determined, allowing for relatively large-scale or high-throughput screening, as discussed herein. For instance, a plurality of cells may be determined for specific phenotypes (for example, the expression of a suitable protein), and cells with a desirable phenotype may also be determined genotypically.

In some cases, relatively large numbers of cells may be determined. For example, depending on the magnification, a single field of view may contain relatively large numbers of cells (for example, at least 10, at least 100, at least 1.000, at least 10,000, at least 100,000, etc. cells). In addition, a sample may be larger than a single field of view (e.g., especially at relatively high magnifications), and multiple images of different portions of a sample may be acquired, e.g., manually or automatically (for example, using computer control). This may allow even larger numbers of cells to be studied via the use of more than one field of view, for example, at least 10, at least 100, at least 1,000, at least 10,000, at least 100,000, at least 1,000,000, at least 10,000,000, etc. cells. For instance, an overall image of a sample may be assembled using multiple fields of views (for example, taken simultaneously or near-simultaneously) to produce an image; for example, at least 2, at least 3, at least 5, at least 7, at least 10, at least 15, at least 20, at least 30, at least 50, at least 75, or at least 100 images may be acquired at different fields of views (e.g., corresponding to different portions of a sample) to produce the overall image. Thus, the sample may, in some cases, be substantially larger than a single field of view. For example, a sample may have an area of at least about 0.01 cm², at least about 0.03 cm², at least about 0.1 cm², at least about 0.3 cm², at least about 1 cm², at least about 3 cm², or at least about 10 cm², etc.

In some embodiments, multiple images may be taken for the same field of view. For example, at least 2, at least 3, at least 5, at least 7, at least 10, at least 15, at least 20, at least 30, at least 50, at least 75, or at least 100 images may be acquired for the same field of view.

In some cases, multiple images may be taken at each of the fields of view imaged within a sample, in one set of embodiments. In some embodiments, different wavelengths may be used. For example, in some cases, images may be collected, for example, with different illumination sources, and captured using different optical filters so as to produce different colors of images that probe the presence of different fluorescent compounds. Thus, in some embodiments, multiple images may be taken at different wavelengths, e.g., to view the images in different colors (for example, red-green-blue, red-yellow-blue, cyan-magenta-yellow, or the like).

In some embodiments, these images may be collected at defined time intervals so as to create time-lapse images of the sample. This may be useful, for example, to determine properties that change with time, e.g., the growth of cells. For example, an image (or a plurality of images) may be acquired at different points in time, e.g., with a periodicity of about 5 seconds, about 10 seconds, about 15 seconds, about 30 seconds, about 1 minute, about 2 minutes, about 3 minutes, about 5 minutes, about 10 minutes, about 15 minutes, about 20 minutes, about 30 minutes, about 1 hour, about 2 hours, about 3 hours, about 4 hours, about 1 day, or the like.

Similarly, in some embodiments, images may be collected after different treatments of the same sample.

In addition, in some embodiments, multiple images may be collected with different imaging modalities, e.g. super-resolution optical microscopy, conventional epi-fluorescence microscopy, confocal microscopy, etc., including those described herein. Such images may be combined, in some cases, to create high content optical measurements of the properties of the cells.

The cells may be any suitable cells. for example, bacterial cells (e.g., E. coli), mammalian cells (e.g., human or non-human cells), eukaryotic cells, prokaryotic cells, yeast cells, or other types of cells. The cells may arise from any suitable source, for example, a cell culture. In some cases, the cells may be taken from a tissue sample, e.g., from a biopsy, artificially grown or cultured, etc. In some cases, the cells are genetically engineered. In some cases, a tissue sample may be analyzed. In certain embodiments, a plurality of cells may be transfected as discussed herein, and the resulting phenotypes of the cells determined.

It should also be understood that certain embodiments of the invention may be directed to systems that do not necessarily include cells. For example, in some cases, one or more of the cells may be artificial cells, for example, wells that contain biological components that can express the desired phenotype. For example, in certain embodiments, the cells may comprise microfluidic wells or microfluidic droplets that contain enzymatic components required to perform in vitro translation.

In one set of embodiments, the cells may be transfected with a nucleic acid comprising an identification portion or “barcode” of nucleotides, which may be used to distinguish a nucleic acid in one cell from those in other cells. A library of identification portions may be used in certain embodiments, e.g., containing at least 10, at least 10², at least 10³, at least 10⁴, at least 10⁵, at least 10⁶, at least 10⁷, at least 10⁸, etc. unique sequences. The unique sequences may be all individually determined (e.g., randomly), although in some cases, the identification portion may be defined as a plurality of variable portions (or “bits”). e.g., in sequence. For example, an identification portion may include at least 2, at least 3, at least 5, at least 7, at least 10, at least 15, at least 20. at least 25, at least 30, at least 40, or at least 50 variable portions. Each of the variable portions may include at least 2, at least 3, at least 4, at least 5, at least 7, at least 10, at least 15, at least 20, at least 25, at least 30, at least 40, at least 50, or more possibilities.

Thus, for example, an identification portion defined with 22 variable regions and 2 unique possibilities per variable region would define a library of identification portions with 2²²=4,194,304 members. As another non-limiting example, an identification portion may be defined with 10 variable regions and 7 unique possibilities per variable region to define a library of identification portions with 7¹⁰ members. It should be understood that a variable portion may include any suitable number of nucleotides, and different variable portions within an identification portion may independently have the same or different numbers of nucleotides. Different variable regions also may have the same or different numbers of unique possibilities.

For example, a variable portion may be defined having a length of at least 2, at least 3, at least 4, at least 5, at least 7, at least 10, at least 15, at least 20. at least 25, at least 30, at least 40, at least 50, or more nucleotides, and/or a maximum length of no more than 50, no more than 40, no more than 30, no more than 25, no more than 20, no more than 15, no more than 10, no more than 7, no more than 5, no more than 4, no more than 3, or no more than 2 nucleotides. Combinations of these are also possible, e.g., a variable portion may have a length of between 5 and 50 nt. or between 15 and 25 nt, etc. A non-limiting example of a library is illustrated with Readout sequences 1-1, 1-0, 2-1, 2-0, etc. through 22-1 and 22-0, which may be concatenated together (e.g., Readout sequence 1-Readout sequence 2-Readout sequence 3- . . . -Readout 22) to produce an identification sequence (in this non-limiting example, each sequence position 1, 2, . . . 22 may have one of two possibilities, identified with −0 and −1, e.g., sequence position 1 can be either Readout sequence 1-1 or 1-0. sequence position 2 can be cither Readout sequence 2-1 or 2-0, etc.). Similarly, according to certain embodiments, information could also be included in the absence of such sequences. For example, the same information included in the presence of one sequence (e.g. sequence 1-0), could also be determined from the absence of another sequence (e.g., sequence 1-1)

Each readout sequence position may be thought of as a “bit” (e.g., 1 or 0 in this example), although it should be understood that the number of possibilities for each “bit” is not necessarily limited to only 2, unlike in a computer. In other embodiments, as previously discussed, there may be 3 possibilities (i.e., a “trit”), 4 possibilities (i.e., a “quad-bit”), 5 possibilities, etc., instead of only 2 possibilities as in some embodiments. However, the use of bits (of any number of possibilities) to form an identification portion can allow, in some but not all embodiments, the use of codewords, error-detecting codes, error-correcting codes, or the like within the identification portion, for example, as discussed in detail herein.

In some cases, the variable portions of the identification portion may be concatenated together to produce the identification portion. In other cases, however, one or more variable portions may be separated, for example, with constant portions of nucleotides, to produce the identification portion. In addition, in some cases, all of the possible variable portions within a library may be unique, e.g., to minimize confusion. Any method may be used for the concatenation. For example, the portions may be concatenated together using ligation. overlap PCR, oligonucleotide pool synthesis, or other techniques known to those of ordinary skill in the art for joining or concatenating nucleic acids together.

In certain embodiments, all members of a library are produced and/or are used. In other embodiments, however, not all members of a library are necessarily produced and/or used. For example, in some embodiments, e.g., to reduce or eliminate ambiguity or inadvertent reuse, a smaller subset of the library may be used, e.g., less than 75%, less than 50%, less than 40%, less than 30%, less than 20%, or less than 10% of all possible members of a library are produced and/or are used.

In addition, in one set of embodiments, the cells may be transfected with a nucleic acid comprising an expression portion. The expression portion may be any suitable nucleic acid sequence that is suspected of being able to alter the phenotype of a cell. For example, the expression portion may encode a gene, a protein, a regulatory sequence (for example, an operon, a promoter, a repressor, a transcription factor binding site, etc.), a sequence encoding non-coding RNA (for example, miRNA, siRNA, rRNA, tRNA, IncRNA, snoRNA, snRNAs, exRNAs, piRNA, tsRNA, rsRNA, shRNA, Cas9 guide RNA, etc.), or the like. In some cases, the expression portion may be part of the same nucleic acid comprising an identification portion; in other cases, however, the expression portion may be part of a different nucleic acid.

In certain embodiments, there may be more than one possibility for the expression portion. For example, a library of nucleic acids may be prepared where there are at least two possibilities for the expression portion. In certain cases, there may be at least 10, at least 10², at least 10³, at least 10⁴, at least 10⁵, etc. possibilities for an expression portion. In addition, in some cases, more than one expression portion may be present (for example, encoding two genes, a gene and a noncoding nucleic acid sequence such as an siRNA sequence, or the like).

As an example, a plurality of distinguishable nucleic acids may be prepared using one or more identification portions (such as those described above) and one or more expression portions. It should be understood, however, that the number of possible identification portions need not equal the number of possible expression portions, i.e., there may be some redundancy involved.

In certain embodiments, the expression portion and the identification portions are combined randomly within the nucleic acids, e.g., to form a nucleic acid library. In other embodiments, the expression portion and the identification portion are combined deterministically within the nucleic acids, e.g., to form a nucleic acid library.

In some cases, the association between identification portion and expression portion can be determined by sequencing the nucleic acids. Any technique may be used for sequencing, for example, Sanger sequencing, high-throughput sequencing, next generation sequencing, nanopore sequencing, sequencing by ligation, sequencing by synthesis, etc. Those of ordinary skill in the art will be aware of different techniques for sequencing nucleic acids.

To facilitate sequencing, in some embodiments, each unique associated expression portion and identification portion may additionally be associated with another nucleotide sequence, for example, of at least 5, at least 10, at least 15, between 5 and 50, between 10 and 100, between 5 and 30 nucleotides, or the like. For example, the unique nucleotide sequence may be used to match reads in, for example, high-throughput sequencing. In some cases, the identification portion and the expression portion may contain a selective factor that allows selection for properly combined identification portions and expression portions. As a non-limiting example, the identification portion could be associated with, for example, an antibiotic resistance gene and the expression portion could, for example, be associated with a plasmid replication origin. Thus, for example, transfected cells can be selected or sorted in certain embodiments, for example, by antibiotic selection using the resistance gene.

In certain embodiments, for example, an expression portion may comprise a gene. In some cases, more than one possibility for the gene may be present in a library. For instance, one possibility may represent a wild-type form while another possibility may represent a diseased form, a genetic variant, a mutant form, or the like of a protein. In some cases, there may be a gene and all (or a subset of all) possible single amino acid substitutions and/or all possible single amino acid insertions of the gene and/or all possible single amino acid deletions of the gene present within the library. In addition, this may be extended even further. For example, there may also be all possible (or a subset of all) two amino acid substitutions of the gene and/or all possible two amino acid insertions of the gene and/or all possible two amino acid deletions of the gene (and these two amino acid substitutions and/or insertions and/or deletions may be consecutive or nonconsecutive). This can be extended to three, four, five, etc. amino acids as well in certain instances.

In other embodiments, the expression portion may represent a property of an external environment to which the cell is exposed. As non-limiting examples, the expression portion may correspond to viral vectors printed onto discrete regions of a cell culture substrate, with the spatial location of the cells determining which of the expression portions to which they are exposed. Similarly, in another example, the expression portion could represent small molecules added individually to the cells (for example, if the cells are in wells).

It should be understood that although the number of expression portions and/or identification portions may be relatively large number of possibilities (for example, millions), this is readily achievable by one of ordinary skill in the art using technologies such as computers and automated nucleic acid synthesis machines (many of which are commercially available), as well as techniques such as solid-phase synthesis and/or isothermal assembly (see, e.g., Example 4) and/or error-prone PCR and/or ligating or otherwise assembling by for example overlap PCR multiple variable regions combinatorially. Similarly, a correspondingly relatively large number of unique identification portions may be correlated with such large numbers of possibilities for the expression portions, for example, through the use of relatively small numbers of suitable variable regions and unique “bits” that can be produced for each. Accordingly, a library of nucleic acids (e.g., each containing an identification portion and an expression portion) may be prepared, e.g., containing at least 10, at least 10², at least 10³, at least 10⁴, at least 10⁵, at least 10⁶, at least 10⁷, at least 10⁸, etc. unique members.

In one set of embodiments, nucleic acids from the library of nucleic acids may be transfected or otherwise introduced into a cell. Any suitable technique may be used to introduce the nucleic acid. In one set of embodiments, the nucleic acids may be incorporated into plasmids that may be taken up by the cells. Other methods of transfection of nucleic acids into cells include, but are not limited to, calcium phosphate (e.g., tricalcium phosphate), electroporation, cell squeezing, mixing a cationic lipid with the material to produce liposomes which fuse with the cell membrane, or the like. Additional non-limiting examples of suitable methods include dendrimers, cationic polymers, lipofection, FuGENE, sonoporation, optical transfection, protoplast fusion, impalefection, the gene gun, magnetofection, particle bombardment, viral infection, or the like.

In certain embodiments, the nucleic acids may be introduced to the cells such that at least 50% of the cells have only 0 or 1 nucleic acids introduced therein, e.g., transfected. In some cases, at least 60%, at least 70%, at least 80%, at least 85%, at least 90%, or at least 95% of the cells may have only 0 or 1 nucleic acids introduced therein. This may be achieved, for example, using suitable dilution techniques, suitable cell sorting techniques, or through the use of other techniques such as microfluidic droplets. In other cases, the percent of transfected cells may be smaller, such as less than 50%, less than 20%, less than 10%, less than 1%. In some embodiments, the non-transfected cells may be removed. Non-limiting examples of cell removal include treatment with a chemical (such as an antibiotic) that, for example, kills or prevents from dividing the non-transfected cells. In another example, some or all of the non-transfected cells may be sorted from the transfected cells using, for example fluorescence activated cell sorting and/or other suitable cell sorting or microfluidic techniques.

In certain embodiments, the identification portion and the expression portion may be combined onto a single source, e.g. a single plasmid. In other embodiments, these portions may be provided to the cell in separate sources, e.g. two different plasmids, or two different viral delivery vehicles. Other examples of introducing a nucleic acid into a cell are disclosed herein, and the methods of introduction may be the same or different. In some embodiments, the expression portion could represent an external stimulus provided to the sample, such as those provided via small molecules.

The combination of the identification portion and the expression portion, whether it is on the same or different vehicles, e.g., plasmids, can be determined, for example, randomly or deterministically. For example, a given protein mutant can be assigned to a given barcode, and the plasmids expressed each of these items co-transfected into a single cell culture. As another example, a library of protein mutants and a library of given barcodes can be combined with each cell obtaining a random combination of the two. In some embodiments, the specific association between the identification and the expression portions can be measured with any of a variety of techniques. For example, PCR may be used to amplify a portion of a plasmid containing both the identification and the expression portions, and then sequencing approaches, included next-generation sequencing methods, can be used to identify which identification region occurs with which expression portion via direct sequencing of this PCR product. Those of ordinary skill in the art will be aware of other techniques that can be used to sequence the nucleic acids, e.g., containing the identification portion and the expression portion.

The cells may be analyzed to determine their phenotype, in another aspect. The phenotype may be determined using any suitable technique, for example, using optical techniques, through analysis of cell behavior, or the like. Specific examples include, but are not limited to, microscopy or other optical techniques such as light microscopy, fluorescence microscopy, confocal microscopy, near-field microscopy, two-photon microscopy, or phase contrast microscopy, or other techniques described herein. In some cases, super-resolution techniques may be used, including any of those described herein. In some cases, the phenotype can be probed by other techniques, such as atomic force microscopy or patch clamping. In some cases, both microscopy and another technique can be used in combination for determining the phenotype.

Examples of phenotype that may be determined include, but are not limited to, the morphology of a cell (e.g., shape, size, visual appearance, organelles, etc.), certain characteristics of cell motility (for example, speed, persistence, chemotaxis behavior, etc.), certain characteristics of inter-cellular interactions (e.g. cell to cell adhesion, cell to cell avoidance etc.), or certain subcellular characteristics (for example position of a protein or nucleic acid, diffusion of protein or nucleic acids, binding of two or more proteins and/or nucleic acids, etc.). In certain embodiments, the cells are present on a substrate, for example, suitable for culturing and/or imaging cells. For example, the substrate may be glass, silicon, plastic (for example, polystyrene, polypropylene, polycarbonate, etc.), or the like. In some cases, at least a portion of the substrate may be at least partially optically transparent. The substrate may also be untreated or treated in some fashion to facilitate cell attachment.

In some embodiments, phenotypes that may be determined include all, or at least a portion, of the transcriptome of the cells. A variety of techniques may be used to determine transcriptomes including, but not limited to, smFISH, MERFISH, or other techniques such as those described herein. See also U.S. patent application Ser. No. 15/329,683 or Int. Pat. Apl. Pub. No. WO 2016/018960. each incorporated herein by reference in its entirety. In some cases, the transcriptome may be determined spatially within one or more cells.

In addition, in some cases, phenotypes that may be determined include all, or at least a portion, of the chromosome of the cells, and/or agents such as proteins or RNA that may be bound to or otherwise associated with the chromosome of the cells. For example, concentrations, spatial positions, activities, associations, etc. of the chromosomes and/or other associated agents may be determined, according to certain embodiments of the invention. In some cases, the chromosomes may be determined spatially within one or more cells. Non-limiting examples of techniques that may be used to determine chromosomes include multiplexed DNA FISH or CASFISH.

In addition, in some cases, phenotypes that may be determined include all, or at least a portion, of the proteome of the cells. A variety of techniques may be used to determine proteomes include antibody labeling, sequential antibody labeling, multiplexed antibody imaging, or other multiplexed protein imaging techniques. For example, concentrations, spatial positions, activities, associations, etc. of the proteins and/or other associated agents may be determined.

In certain embodiments, one or more markers may be determined within the cell to determine a phenotype. For example, the marker may be indicative for a certain cell protein, nucleic acid, morphological characteristic, or the like, or the marker may be indicative of cell behavior. In addition, the marker may be one that can be visually determined in some cases. For example, the marker may be fluorescent, or may alter fluorescence of another fluorescent entity within the cell (for example, via enhancement or quenching). The marker may also be a dye or may change color in some embodiments. Accordingly, differences in intensity, wavelength, frequency, position, distribution, or the like between cells in an image may be determined to determine phenotypes of the cells. Other methods of determining a marker may also be used in some cases; for example, the marker may be radioactive. Many such markers may be obtained commercially.

Moreover, it should be understood that these measurements are not mutually exclusive. Any combination of these measurements can be performed in a single sample. Moreover, such measurements may be repeated in some embodiments, e.g., for the same sample. For instance, the measurements may be repeated to ensure validity or reduce potential errors (e.g., measurement errors), or the measurements may be repeated after exposure to various stimuli or conditions, such as treatment with different nutritional sources, small molecules, or other suitable agents that may interact with the cells.

In some cases, the phenotype of a cell may be altered by application of an expression portion, e.g., as discussed above, that may be expressed in some form by the cell to alter its phenotype. For example, an expression portion that encodes a protein to the cell may be added, and the cell may express the protein. If different proteins are encoded in different cells, then the cells may exhibit different phenotypes, which can be determined as noted above. Thus, for instance, a plurality of cells may be transfected or otherwise introduced to a plurality of different expression portions, and then the cells studied to determine the effects the different expression portions have had on their phenotype.

The expression portion, in some embodiments, may produce the marker (for example, if the marker is a fluorescent protein), or the expression portion may produce a product (for example, a protein or a nucleic acid sequence) that can interact with a marker in some fashion, directly or indirectly, which results in a determinable change in phenotype (for example, that can be identified using a suitable fluorescent compound), or in other cases, the expression portion may otherwise change the abundance of the marker, directly or indirectly (for example, if the expression portion is a promoter, gene regulatory element, shRNA, guide RNA, etc.).

In certain embodiments, the phenotype may be produced from a specific combination of multiple sequences. For example, the phenotype might represent the overexpression of individual pairs of proteins. As another non-limiting example, the phenotype could be generated by a mutation to the host genome as well as overexpression of a separate protein. It should be understood that the expression portion represents the unique combination of the elements necessary to express the full phenotype and the number of elements need not be limited to two, as described here.

In some aspects, cells may be immobilized or fixed to a substrate, e.g., prior to determining genotype as discussed below. In some cases, immobilization or fixing of the cells may occur after determination of phenotype. This may be useful according to certain embodiments, for example, to correlate the phenotype of the cells within an image with the subsequent genotype of the cells (e.g., determined as discussed below). The cells can also be fixed in some embodiments before measuring the phenotype instead of after measuring the phenotype and before measuring the genotype.

Those of ordinary skill in the art will be aware of systems and methods for fixing or otherwise immobilizing cells on a substrate. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE). See also U.S. Pat. Apl. Ser. No. 62/419.033, incorporated herein by reference in its entirety.

In one aspect, the genotype of the cells are determined. This can be performed, for example, after determining their phenotype as discussed above. A variety of different techniques for determining the genotype of cells may be used, for example, FISH, smFISH, MERFISH, in situ hybridization, multiplexed FISH, CASFISH, or other techniques known to those of ordinary skill in the art. These approaches can involve, in some embodiments, the direct hybridization to the identification portion, or molecules generated via the host cell from that portion. It can also involve, in certain instances, binding of separate adaptor entities, which in turn bind directly to the identification portion or molecules generated from it. Additional non-limiting examples of techniques include those disclosed in U.S. patent application Ser. No. 15/329,683 or Int. Pat. Apl. Pub. No. WO 2016/018960, each incorporated herein by reference in its entirety.

In one set of embodiments, the determination of the genotype of the cells may be facilitated by determining an identification portion of a nucleic acid within the cells. For example, nucleic acids comprising an identification portion and an expression portion may have been introduced into the cells; the expression portion may have led to different phenotypes as discussed above. However, it would also be important to know which nucleic acids were introduced into which cells, thereby allowing an understanding between the observed phenotypes and the genotypes leading to those phenotypes. By determining the identification portion within the cells, as discussed herein, the identity of the nucleic acid contained within each cell may be determined, and thus a specific expression portion may also be determined, e.g., if the nucleic acid comprises the identification portion and the expression portion on the same individual nucleic acid.

As a non-limiting example, in one set of embodiments, the cells may be sequentially exposed to nucleic acid probes able to bind to different portions of the identification portion, or molecules, such as RNA, expressed by the cell from this identification portion, for example, nucleic acid probes comprising a target sequence (e.g., that is able to bind to at least a portion of the identification portion, in some cases specifically) and a read sequence (e.g., which may be “read” in some fashion to determine binding), and binding of the nucleic acid probes within the cells may be determined. For example, the cells may be exposed to secondary nucleic acid probe may contain a recognition sequence able to bind to or hybridize with a read sequence, and which may contain a signaling entity. By determining signaling entities within images (and in some cases, inactivating the signaling entities between images and exposure to different nucleic acid probes), the identification portions of the cells may be determined.

As discussed herein, a variety of nucleic acid probes may be used to determine one or more nucleic acids within a cell. The probes may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components may also be present within the nucleic acid probes, e.g., as discussed below. In some embodiments, the nucleic acid probes can be created from other components, e.g. protein or other small molecules, or may represent a combination of these components with nucleic acids such as DNA, RNA, LNA, PNA, or the like.

The nucleic acid probes may be introduced into the cells using any suitable method. In some cases, the cells may be sufficiently permeabilized such that the nucleic acid probes may be introduced into the cells by flowing a fluid containing the nucleic acid probes around the cells. In some cases, the cells may be sufficiently permeabilized as part of a fixation process; in other embodiments, cells may be permeabilized by exposure to certain chemicals such as ethanol, methanol, Triton, or the like. In addition, in some embodiments, techniques such as electroporation or microinjection may be used to introduce nucleic acid probes into the cells.

The determination of nucleic acids within the cells may be qualitative and/or quantitative. In addition, the determination may also be spatial, e.g., the position of the nucleic acid within the cells may be determined in two or three dimensions. In some embodiments, the positions, number, and/or concentrations of nucleic acids within the cells may be determined.

Thus, certain aspects of the present invention are generally directed to nucleic acid probes that are introduced into a cell. The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target nucleic acid, in some cases specifically. When introduced into a cell or other system, the target system may be able to bind to a specific target nucleic acid (e.g., an mRNA, or other nucleic acids as discussed herein). In some cases, the nucleic acid probes may be determined using signaling entities (e.g., as discussed below), and/or by using secondary nucleic acid probes able to bind to the nucleic acid probes (i.e., to primary nucleic acid probes). The determination of such nucleic acid probes is discussed in detail below.

In some cases, more than one type of (primary) nucleic acid probe may be applied to the cells, e.g., simultaneously. For example. there may be at least 2, at least 5, at least 10, at least 25, at least 50, at least 75, at least 100, at least 300, at least 1,000, at least 3,000, at least 10,000, or at least 30,000 distinguishable nucleic acid probes that are applied to the cells, e.g., simultaneously or sequentially.

The target sequence may be positioned anywhere within the nucleic acid probe (or primary nucleic acid probe or encoding nucleic acid probe). The target sequence may contain a region that is substantially complementary to a portion of a target nucleic acid. In some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc. Typically, complementarity is determined on the basis of Watson-Crick nucleotide base pairing.

The target sequence of a (primary) nucleic acid probe may be determined with reference to a target nucleic acid suspected of being present within a cell. For example, a target nucleic acid to a protein may be determined using the protein's sequence, by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe, such as a primary nucleic acid probe, may also comprise one or more “read” sequences. However, it should be understood that read sequences are not necessary in all cases. In some embodiments, the nucleic acid probe may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the nucleic acid probe. If more than one read sequence is present, the read sequences may be positioned next to each other, and/or interspersed with other sequences. In some embodiments, the read sequence is contained within the identification portion and/or in RNA expressed from the identification portion.

The read sequences, if present, may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides between 10 and 200 nucleotides, or between 25 and 35 nucleotides, between 10 and 300 nucleotides, etc.

The read sequence may be arbitrary or random in some embodiments. In certain cases, the read sequences are chosen so as to reduce or minimize homology with other components of the cell, e.g., such that the read sequences do not themselves bind to or hybridize with other nucleic acids suspected of being within the cell. In some cases, the homology may be less than 10%, less than 8%, less than 7%, less than 6%, less than 5%, less than 4%, less than 3%, less than 2%, or less than 1%. In some cases, there may be a homology of less than 20 basepairs, less than 18 basepairs, less than 15 basepairs, less than 14 basepairs, less than 13 basepairs, less than 12 basepairs, less than 11 basepairs, or less than 10 basepairs. In some cases, the basepairs are sequential.

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2ⁿ−1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15. etc. or more read sequences. In some cases, a population of nucleic acid probes may each contain the same number of read sequences, although in other cases, there may be different numbers of read sequences present on the various probes. As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein.

In addition, in some embodiments, the nucleic acid probes (and/or their corresponding, complimentary sites on, for example, the encoding probes or RNA that may be transcribed from the identification portion, or on the identification portion itself), in certain embodiments, may be made using only 2 or only 3 of the 4 bases, such as leaving out all the “G”s or leaving out all of the “C”s within the probe. Sequences lacking either “G”s or “C”'s may form very little secondary structure in certain embodiments, and can contribute to more uniform, faster hybridization.

In some embodiments, the nucleic acid probe may contain a signaling entity. It should be understood that signaling entities are not required in all cases, however; for instance, the nucleic acid probe may be determined using secondary nucleic acid probes in some embodiments, as is discussed in additional detail below. Examples of signaling entities that can be used are also discussed in more detail below.

Other components may also be present within a nucleic acid probe as well. For example, in one set of embodiments, one or more primer sequences may be present, e.g., to allow for enzymatic amplification of probes. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally-occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In addition, the components of the nucleic acid probe may be arranged in any suitable order. For instance, in one embodiment, the components may be arranged in a nucleic acid probe as: primer-read sequences-targeting sequence-read sequences-reverse primer. The “read sequences” in this structure may each contain any number (including 0) of read sequences, so long as at least one read sequence is present in the probe. Non-limiting example structures include primer-targeting sequence-read sequences-reverse primer, primer-read sequences-targeting sequence-reverse primer, targeting sequence-primer-targeting sequence-read sequences-reverse primer, targeting sequence-primer-read sequences-targeting sequence-reverse primer, primer-target sequence-read sequences-targeting sequence-reverse primer, targeting sequence-primer-read sequence-reverse primer, targeting sequence-read sequence-primer, read sequence-targeting sequence-primer, read sequence-primer-targeting sequence-reverse primer, etc. In addition, the reverse primer is optional in some embodiments, including in all of the above-described examples.

After introduction of the nucleic acid probes into a cell, the nucleic acid probes may be directly determined by determining signaling entities (if present), and/or the nucleic acid probes may be determined by using one or more secondary nucleic acid probes, in accordance with certain aspects of the invention. As mentioned, in some cases, the determination may be spatial, e.g., in two or three dimensions. In addition, in some cases, the determination may be quantitative, e.g., the amount or concentration of a primary nucleic acid probe (and of a target nucleic acid) may be determined. Additionally, the secondary probes may comprise any of a variety of entities able to hybridize a nucleic acid, e.g., DNA, RNA, LNA, and/or PNA, etc., depending on the application. Signaling entities are discussed in more detail below.

A secondary nucleic acid probe may contain a recognition sequence able to bind to or hybridize with a read sequence of a primary nucleic acid probe. In some cases, the binding is specific, or the binding may be such that a recognition sequence preferentially binds to or hybridizes with only one of the read sequences that are present. The secondary nucleic acid probe may also contain one or more signaling entities. If more than one secondary nucleic acid probe is used, the signaling entities may be the same or different.

The recognition sequences may be of any length, and multiple recognition sequences may be of the same or different lengths. If more than one recognition sequence is used, the recognition sequences may independently have the same or different lengths. For instance, the recognition sequence may be at least 5, at least 10, at least 15, at least 20, at least 25, at least 30, at least 35, at least 40, or at least 50 nucleotides in length. In some cases, the recognition sequence may be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30, between 20 and 40, or between 25 and 35 nucleotides, etc. In one embodiment, the recognition sequence is of the same length as the read sequence. In addition, in some cases, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to a read sequence of the primary nucleic acid probe.

As mentioned, in some cases, the secondary nucleic acid probe may comprise one or more signaling entities. Examples of signaling entities are discussed in more detail below. As discussed, in certain aspects of the invention, nucleic acid probes are used that contain various “read sequences.” For example, a population of primary nucleic acid probes may contain certain “read sequences” which can bind certain of the secondary nucleic acid probes, and the locations of the primary nucleic acid probes are determined within the cells using secondary nucleic acid probes, e.g., which comprise a signaling entity. As mentioned, in some cases, a population of read sequences may be combined in various combinations to produce different nucleic acid probes, e.g., such that a relatively small number of read sequences may be used to produce a relatively large number of different nucleic acid probes. Thus, in some cases, a population of primary nucleic acid probes (or other nucleic acid probes) may each contain a certain number of read sequences, some of which are shared between different primary nucleic acid probes such that the total population of primary nucleic acid probes may contain a certain number of read sequences. A population of nucleic acid probes may have any suitable number of read sequences. For example, a population of primary nucleic acid probes may have 1, 2, 3, 4, 5, 6, 7, 8, 9, 10, 11, 12, 13, 14, 15, 16, 17, 18, 19, 20 etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32. no more than 24, no more than 20, no more than 16, no more than 15, no more than 14, no more than 13, no more than 12, no more than 11. no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially producing a relatively large number of nucleic acid probes from a relatively small number of read sequences, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16. 32, etc. or more read sequences, or any other suitable number of read sequences described herein, depending on the application. If each of the primary nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of primary nucleic acid probes, up to 10 probes may be separately identified. For example, one of ordinary skill in the art would understand that, for k read sequences in a population with n read sequences on each probe, up to

$$( \text{?} )$$

$$\text{?} \text{indicates text missing or illegible when filed}$$

different probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance example, some probes may contain 2 read sequences while other probes may contain 3 read sequences.

In some aspects, the read sequences and/or the pattern of binding of nucleic acid probes within the cells may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases). Multiple rounds of binding determinations, e.g., using different nucleic acid probes, can then be used to create a “codeword,” e.g., for that spatial location. In some embodiments, the codeword may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g. where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the read sequences or binding patterns of the nucleic acid probes are assigned such that not every possible combination is assigned.

For example, if 4 read sequences are possible and a primary nucleic acid probe contains 2 read sequences, then up to 6 primary nucleic acid probes could be identified; but the number of primary nucleic acid probes used may be less than 6. Similarly, for k read sequences in a population with n read sequences on each primary nucleic acid probe,

$$( \text{?} )$$

$$\text{?} \text{indicates text missing or illegible when filed}$$

different probes may be produced, but the number of primary nucleic acid probes that are used may be any number more or less than

$$( \text{?} ) .$$

$$\text{?} \text{indicates text missing or illegible when filed}$$

In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected up to 2ⁿ different targets may be possible for n rounds of probes, but the number of nucleic acid targets that are actually used may be any number less than 2ⁿ. For example, if in each round, each target can give more than two possible outcomes, such as being detected in different color channels, more than 2ⁿ (e.g. 3ⁿ, 4ⁿ . . . ) different targets may be possible for n rounds of probes. In some cases, the number of nucleic acid targets that are actually used may be any number less than this number. In addition, these may be randomly assigned, or assigned in specific ways to increase the ability to detect and/or correct errors.

For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. In certain cases, the Hamming distance may be at least 2, at least 3, at least 4, at least 5, at least 6, or the like. In addition, in one set of embodiments, the assignments may be formed as a Hamming code, for instance, a Hamming(7, 4) code, a Hamming(15, 11) code, a Hamming(31, 26) code, a Hamming(63, 57) code, a Hamming(127, 120) code, etc. In another set of embodiments, the assignments may form a SECDED code, e.g., a SECDED(8,4) code, a SECDED(16,4) code, a SCEDED(16, 11) code, a SCEDED(22, 16) code. a SCEDED(39, 32) code, a SCEDED(72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, a code with the same error correcting properties of the SECDED code may be formed by using only binary words that contain a fixed number of ‘1’ bits, such as 4, to encode the targets. In another set of embodiments, the assignments may represent a subset of the possible values taken from codes described above for the purpose of addressing asymmetric readout errors. For example, in some cases, a code in which the number of ‘1’ bits may be fixed for all used binary words may eliminate the biased measurement of words with different numbers of ‘1’s when the rate at which ‘0’ bits are measured as ‘1’s or ‘1’ bits are measured as ‘0’s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc. Non-limiting examples of such codes include the Reed-Solomon erasure codes and generalizations thereof.

In addition, the code can also be selected in some embodiments through random selection of a sub-set of all possible codewords. For example, a random subset of binary codewords of length n code be selected. In some cases, these codewords can be separated by Hamming distances, i.e. the number of bits that must be flipped to convert one into another, so that some of the used codewords maintain some error robust or correcting abilities. In some embodiments, approaches such as next-generations sequencing can be used to measure the random subset of codewords used and error robustness and error correction could be applied selectively on the codewords that satisfy the constraints necessary for these properties.

As discussed above, in certain aspects, signaling entities are determined, e.g., to determine nucleic acid probes and/or to create codewords. In some cases, signaling entities within the cells may be determined, e.g., spatially, using a variety of techniques. In some embodiments, the signaling entities may be fluorescent, and techniques for determining fluorescence within the cells, such as fluorescence microscopy or confocal microscopy, may be used to spatially identify the positions of signaling entities within a cell. In some cases, the positions of entities within the cells may be determined in two or even three dimensions. In addition, in some embodiments, more than one signaling entity may be determined at a time (e.g., signaling entities with different colors or emissions), and/or sequentially.

In addition, in some embodiments, a confidence level for the identified nucleic acid target may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3., greater than about 0.5 greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the identified nucleic acid target is greater than an internal standard or false positive control by about 0.01, about 0.03, about 0.05, about 0.1, about 0.3. about 0.5, about 1, about 3, about 5, about 10, about 30, about 50, about 100, about 300, about 500, about 1000, or any other suitable value

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities optically, e.g., using fluorescence microscopy. In some cases, the spatial positions may be determined at super resolutions, or at resolutions better than the wavelength of light or the diffraction limit. Non-limiting examples include STORM (stochastic optical reconstruction microscopy), STED (stimulated emission depletion microscopy). NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy). Super-resolution optical fluctuation imaging (SOFI), or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or Int. Pat. Apl. Pub. No. WO 2013/090360. published Jun. 20. 2013, entitled “High Resolution Dual-Objective Microscopy,” by Zhuang, et al., each incorporated herein by reference in their entireties.

As an illustrative non-limiting example, in one set of embodiments, the cells may be imaged with a high numerical aperture, oil immersion objective with 100× magnification and light collected on an electron-multiplying CCD camera. In another example, the cells could be imaged with a high numerical aperture, oil immersion lens with 40× magnification and light collected with a wide-field scientific CMOS camera. With different combinations of objectives and cameras, a single field of view may correspond to no less than 40×40 microns, 80×80 microns, 120×120 microns, 240×240 microns, 340×340 microns, or 500×500 microns, etc. in various non-limiting embodiments. Similarly, a single camera pixel may correspond, in some embodiments, to regions of the cells of no less than 80×80 nm, 120×120 nm, 160×160 nm, 240×240 nm, or 300×300 nm, etc. In another example, the cells may be imaged with a low numerical aperture, air lens with 10× magnification and light collected with a sCMOS camera. In additional embodiments, the cells may be optically sectioned by illuminating it via a single or multiple scanned diffraction limited foci generated either by scanning mirrors or a spinning disk and the collected passed through a single or multiple pinholes. In another embodiment, the cells may also be illuminated via thin sheet of light generated via any one of multiple methods known to those versed in the art.

In one embodiment, the cells may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In addition, the signaling entity may be inactivated in some cases. For example, in some embodiments, a first secondary nucleic acid probe containing a signaling entity may be applied to the cells that can recognize a first read sequence, then the first secondary nucleic acid probe can be inactivated before a second secondary nucleic acid probe is applied to the cells. If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the cells, or from the nucleic acid probe, etc.), and/or by chemically altering the signaling entity in some fashion, e.g., by photobleaching the signaling entity, bleaching or chemically altering the structure of the signaling entity, e.g., by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe (for example. using tris(2-carboxyethyl)phosphine) and washed away via fluid flow.

In some embodiments, various nucleic acid probes (including primary and/or secondary nucleic acid probes) may include one or more signaling entities. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within the cells at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes are used, the nucleic acid probes may comprise the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswtichable dyes, photoactivatable dyes, fluorescent dyes, metal nanoparticles, semiconductor nanoparticles or “quantum dots”, fluorescent proteins such as GFP (Green Fluorescent Protein), or photoactivabale fluorescent proteins, such as PAGFP, PSCFP, PSCFP2, Dendra, Dendra2, EosFP, tdEos, mEos2, mEos3, PAmCherry, PAtagRFP, mMaple, mMaple2, and mMaple3. Other suitable signaling entities are known to those of ordinary skill in the art. See, e.g., U.S. Pat. No. 7,838,302 or U.S. Pat. Apl. Ser. No. 61/979,436, each incorporated herein by reference in its entirety.

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2 Dnitrobenzyl, biotin phosphoramidite, acrylic phosphoramidite, diethylaminocoumarin, 1-(4,5-dimethoxy-2-nitrophenyl)ethyl, cyclo-dodecyl (dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,4′-dimethoxytrityloxy)butyramidomethyl)phenyl]-ethyl-[2-cyanoethyl-(N,N-diisopropyl)]-phosphoramidite, 1-[2-nitro-5-(6-(N-(4,4′-dimethoxytrityl))-biotinamidocaproamido-methyl)phenyl]-ethyl-[2-cyanoethyl-(N,N-diisopropyl)]-phosphoramidite, or similar linkers.

In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl)phosphine, bis(2-mercaptoethyl) sulfone, N,N′-dimethyl-N,N′-bis(mercaptoacetyl)hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, XmaI, PspAI, DpnI, MboI, MnlI, Eco57I. Ksp6321, DraIII, AhaII, SmaI, MluI, HpaI, ApaI, BclI, BstEII, TaqI, EcoRI, SacI, HindII, HacII, DraII, Tsp509I, Sau3AI, PacI, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be conjugated to the oligonucleotide, while sufficient exposure to an excess of addition, free biotin could “outcompete” the linkage and thereby cause cleavage to occur. In addition, in another set of embodiments, the probes may be removed using corresponding “toe-hold-probes,” which comprise the same sequence as the probe, as well as an extra number of bases of homology to the encoding probes (e.g., 1-20 extra bases, for example, 5 extra bases). These probes may remove the labeled readout probe through a strand-displacement interaction.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

In one set of embodiments, the signaling entity is “switchable,” i.e., the entity can be switched between two or more states, at least one of which emits light having a desired wavelength. In the other state(s), the entity may emit no light, or emit light at a different wavelength. For instance, an entity may be “activated” to a first state able to produce light having a desired wavelength, and “deactivated” to a second state not able to emit light of the same wavelength. An entity is “photoactivatable” if it can be activated by incident light of a suitable wavelength. As a non-limiting example, Cy5, can be switched between a fluorescent and a dark state in a controlled and reversible manner by light of different wavelengths, i.e., 633 nm (or 642nm, 647nm, 656 nm) red light can switch or deactivate Cy5 to a stable dark state, while 405 nm green light can switch or activate the Cy5 back to the fluorescent state. In some cases, the entity can be reversibly switched between the two or more states, e.g., upon exposure to the proper stimuli. For example, a first stimuli (e.g., a first wavelength of light) may be used to activate the switchable entity, while a second stimuli (e.g., a second wavelength of light) may be used to deactivate the switchable entity, for instance, to a non-emitting state. Any suitable method may be used to activate the entity. For example, in one embodiment, incident light of a suitable wavelength may be used to activate the entity to emit light, i.e., the entity is “photoswitchable.” Thus, the photoswitchable entity can be switched between different light-emitting or non-emitting states by incident light, e.g., of different wavelengths. The light may be monochromatic (e.g., produced using a laser) or polychromatic. In another embodiment, the entity may be activated upon stimulation by electric field and/or magnetic field. In other embodiments, the entity may be activated upon exposure to a suitable chemical environment, e.g., by adjusting the pH, or inducing a reversible chemical reaction involving the entity, etc. Similarly, any suitable method may be used to deactivate the entity, and the methods of activating and deactivating the entity need not be the same. For instance, the entity may be deactivated upon exposure to incident light of a suitable wavelength, or the entity may be deactivated by waiting a sufficient time. Typically, a “switchable” entity can be identified by one of ordinary skill in the art by determining conditions under which an entity in a first state can emit light when exposed to an excitation wavelength, switching the entity from the first state to the second state, e.g., upon exposure to light of a switching wavelength, then showing that the entity, while in the second state can no longer emit light (or emits light at a much reduced intensity) when exposed to the excitation wavelength.

In one set of embodiments, as discussed, a switchable entity may be switched upon exposure to light. In some cases, the light used to activate the switchable entity may come from an external source, e.g., a light source such as a laser light source, another light-emitting entity proximate the switchable entity, etc. The second, light emitting entity, in some cases, may be a fluorescent entity, and in certain embodiments, the second, light-emitting entity may itself also be a switchable entity.

In some embodiments, the switchable entity includes a first, light-emitting portion (e.g., a fluorophore), and a second portion that activates or “switches” the first portion. For example, upon exposure to light, the second portion of the switchable entity may activate the first portion, causing the first portion to emit light. Examples of activator portions include, but are not limited to, Alexa Fluor 405 (Invitrogen), Alexa Fluor 488 (Invitrogen), Cy2 (GE Healthcare), Cy3 (GE Healthcare), Cy3B (GE Healthcare). Cy3.5 (GE Healthcare), or other suitable dyes. Examples of light-emitting portions include, but are not limited to, Cy5, Cy5.5 (GE Healthcare), Cy7 (GE Healthcare), Alexa Fluor 647 (Invitrogen), Alexa Fluor 680 (Invitrogen), Alexa Fluor 700 (Invitrogen), Alexa Fluor 750 (Invitrogen), Alexa Fluor 790 (Invitrogen), DiD, DiR, YOYO-3 (Invitrogen), YO-PRO-3 (Invitrogen), TOT-3 (Invitrogen), TO-PRO-3 (Invitrogen) or other suitable dyes. These may linked together, e.g., covalently, for example, directly, or through a linker, e.g., forming compounds such as, but not limited to, Cy5-Alexa Fluor 405, Cy5-Alexa Fluor 488, Cy5-Cy2, Cy5-Cy3, Cy5-Cy3.5, Cy5.5-Alexa Fluor 405, Cy5.5-Alexa Fluor 488, Cy5.5-Cy2. Cy5.5-Cy3, Cy5.5-Cy3.5, Cy7-Alexa Fluor 405, Cy7-Alexa Fluor 488, Cy7-Cy2, Cy7-Cy3, Cy7-Cy3.5, Alexa Fluor 647-Alexa Fluor 405, Alexa Fluor 647-Alexa Fluor 488, Alexa Fluor 647-Cy2, Alexa Fluor 647-Cy3, Alexa Fluor 647-Cy3.5, Alexa Fluor 750-Alexa Fluor 405, Alexa Fluor 750-Alexa Fluor 488, Alexa Fluor 750-Cy2, Alexa Fluor 750-Cy3, or Alexa Fluor 750-Cy3.5. Those of ordinary skill in the art will be aware of the structures of these and other compounds, many of which are available commercially. The portions may be linked via a covalent bond, or by a linker, such as those described in detail below. Other light-emitting or activator portions may include portions having two quaternized nitrogen atoms joined by a polymethine chain, where each nitrogen is independently part of a heteroaromatic moiety, such as pyrrole, imidazole, thiazole, pyridine, quinoine, indole, benzothiazole, etc., or part of a nonaromatic amine. In some cases, there may be 5, 6, 7, 8, 9, or more carbon atoms between the two nitrogen atoms.

Another aspect of the invention is directed to a computer-implemented method. For instance, a computer and/or an automated system may be provided that is able to automatically and/or repetitively perform any of the methods described herein. As used herein, “automated” devices refer to devices that are able to operate without human direction. i.e., an automated device can perform a function during a period of time after any human has finished taking any action to promote the function, e.g. by entering instructions into a computer to start the process. Typically, automated equipment can perform repetitive functions after this point in time. The processing steps may also be recorded onto a machine-readable medium in some cases.

For example, in some cases, a computer may be used to control imaging of the cells, e.g., using fluorescence microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation of signaling entities within the cells, and/or the acquisition of images of the signaling entities. In one set of embodiments, cells may be excited using light having various wavelengths and/or intensities, and the sequence of the wavelengths of light used to excite the cells may be correlated, using a computer, to the images acquired of the cells containing the signaling entities. For instance, the computer may apply light having various wavelengths and/or intensities to the cells to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

In some aspects, the cells are positioned on a microscope. In some cases, the microscope may contain one or more channels, such as microfluidic channels, to direct or control fluid to or from the cells. For instance, in one embodiment, nucleic acid probes such as those discussed herein may be introduced and/or removed from the cells by flowing fluid through one or more channels to or from the cells. In some cases, there may also be one or more chambers or reservoirs for holding fluid, e.g., in fluidic communication with the channel, and/or with the cells. Those of ordinary skill in the art will be familiar with channels, including microfluidic channels, for moving fluid to or from the cells.

The following documents are each incorporated herein by reference in their entireties: U.S. Provisional Patent Application Ser. No. 62/419,033, filed Nov. 8, 2016, entitled “Matrix Imprinting and Clearing”; International Patent Application Ser. No. PCT/US17/60570; International Patent Application Serial No. PCT/US17/60558; International Patent Application Publication No. WO 2016/018960; and International Patent Application Publication No. WO 2016/018963. U.S. Provisional Patent Application Ser. No. 62/511,920, filed May 26, 2017, entitled “High-Throughput. Image-Based Screening of Genetic Variant Libraries,” by Zhuang, et al., is also incorporated herein by reference in its entirety.

The following examples are intended to illustrate certain embodiments of the present invention, but do not exemplify the full scope of the invention.

### EXAMPLE 1

The ability to detect the genetic variant present in a library of surface adhered cells would allow highly versatile image based measurements to be used to determine phenotypes of genetic variant libraries. This example illustrates a new high-throughput, imaging-based screening method that allows the characterization of both phenotype and genotype for pooled populations of genetically diverse cells.

In this example, genetic variants are associated with a barcode composed of a series of short oligonucleotide hybridization sites. After introducing the barcoded genetic variant library into a population of cells and measuring phenotypes with imaging, the cells are fixed and the barcodes are determined using multiplexed error robust fluorescence in situ hybridization (MERFISH), a method that allows tens of thousands of barcodes to be read using combinatorial labeling plus sequential imaging. To test the feasibility and quantify the accuracy of this screening approach, a library was measured with a known phenotype, where half the library members have a fluorescent protein and half do not. In another example, similar methods were used to optimize the brightness and photostability of YFAST, a recently discovered fluorescent protein that becomes fluorescent upon binding to an exogenous chromophore. This allowed efficient screening of 20 million cells containing 160,000 unique barcodes and 60,000 unique mutants, which resulted in the identification of YFAST variants with substantially increased brightness and photostability.

In this example, to screen fluorescent protein mutant libraries for variants with improved properties, such as improved photostability and brightness, a unique barcode was introduced into each plasmid expressing a mutant fluorescent protein.

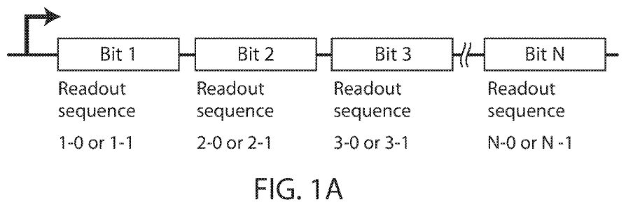

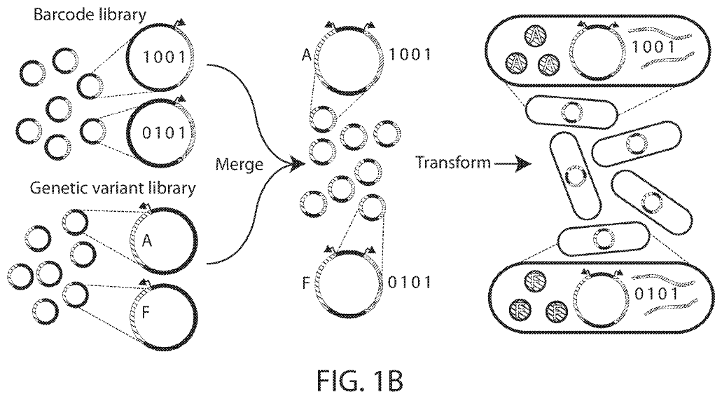

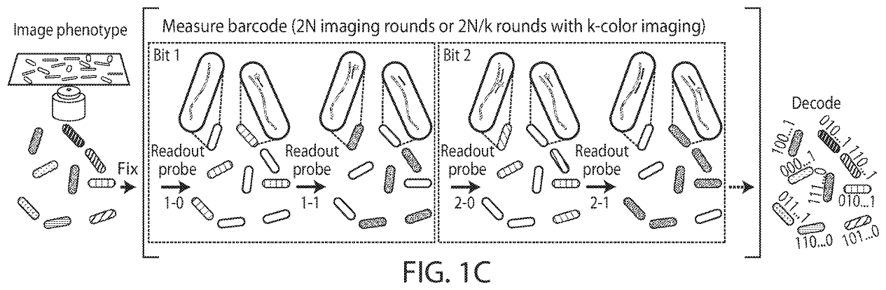

Each barcode was formed from a concatenation of hybridization sites encoding a N-bit binary word (FIG. 1A). Each of the N bit positions can have either a value of 0 or a value of 1 and for each possible bit value, a unique readout sequence was assigned. Altogether, there were 2N unique readout sequences in the barcode: readout sequence 1-0, readout sequence 1-1, readout sequence 2-0. readout sequence 2-1, . . . , readout sequence N-0, readout sequence N-1. For example, the binary word 101 . . . 1 would be encoded in a barcode consisting of readout sequence 1-1, followed by readout sequence 2-0, readout sequence 3-1 , . . . , and finally readout sequence N-1.

To create a barcoded library of genetic variants, the barcode library containing all possible N-bit barcodes and the library of genetic variants were merged so that the barcodes and genetic variants were associated randomly (FIG. 1B). The half of the plasmid containing the genetic variants from the genetic variant library was amplified and assembled with the amplified half of the plasmid containing the barcodes from the barcode library using isothermal assembly and the assembled product is electroporated into E. coli. To reduce the chance of a barcode appearing in the library associated with more than one genetic variant, the final barcoded mutant library was bottlenecked by limiting the number of cells to between 1% and 10% of the total barcode diversity of 2. To determine which barcode was associated with which genetic variant, the plasmids were extracted from the E. coli culture and sequenced by next generation sequencing to construct a look-up table. Still, a small probability remained that the same barcode appears associated with multiple mutants in a library. This situation was detected in the sequencing results and the affected barcodes are removed in further analysis. Within the E. coli cells, the fluorescent protein and the barcode RNA were expressed from the plasmid, allowing the brightness and photostability of the fluorescent protein to be measured along with the identity of the barcode.

To screen the barcoded protein mutant library, the cells were adhered to a glass coverslip and their phenotypes, such as fluorescence intensity and photobleaching rate, were imaged while the cells were still alive (FIG. 1C). Then, the cells were fixed in methanol, without removing them from the microscope, and the barcode was read out using high-throughput imaging, such as multiplexed error-robust fluorescence in situ hybridization (MERFISH).

During the barcode readout process, multiple hybridization rounds were used and fluorescently labeled readout probes complementary to each readout sequence on the barcode were hybridized in each round to detect which readout sequences are present in which cells. First, readout probe 1-0, complementary to readout sequence 1-0, was introduced. It hybridizes to cells that contain readout sequence 1-0. namely the cells containing barcodes whose first bit is “0”, causing those cells to become brightly fluorescent. All the cells were imaged and then the dye, attached to the readout probe by a disulfide bond, was reductively cleaved by TCEP (tris(2-carboxyethyl)phosphine) to make all cells non-fluorescent. Then, readout probe 1-1 was hybridized. Since every barcode contained either readout sequence 1-0 or readout sequence 1-1, the cells that did not become fluorescent in the first round should now become fluorescent. The value for bit 1 for each cell was then assigned based on the fluorescence intensity ratio between probe 1-0 and probe 1-1. The dye was then cleaved. This process was iterated until all N bits were probed. To reduce the number of hybridization rounds, three color imaging was used to allow three probes with spectrally distinct fluorescent dyes to be hybridized and imaged simultaneously.

Since each cell expresses many copies of its corresponding barcode RNA, the fluorescence signal was very bright, and hence the readout error rate for each bit is very small, an error correcting code was unnecessary and all 2^{N} possible barcodes could be used. But, as described above, to avoid a barcode appearing paired with multiple mutants in the same library, the number of unique library members was restricted to be between 1% and 10% the total barcode diversity of 2^{N} by bottlenecking. Still, with this binary encoding scheme, the number of possible binary words scaled exponentially with the number of bits, allowing millions of unique barcodes to be measured with only tens of hybridization rounds.

FIG. 1 shows a high-throughput, image-based screening method using massively multiplexed fluorescence in situ hybridization. FIG. 1A shows a schematic depiction of the barcode. Each barcode was formed from a concatenation of bit-encoding nucleotide sequences where each bit position has either the sequence corresponding to a “0” or a “1.” FIG. 1B shows a schematic depiction of library construction. The library of barcodes was merged with a library of genetic variants and transformed into bacteria. The correspondence between the barcodes and genetic variants was determined by sequencing.

FIG. 1C is a schematic diagram of the image-based phenotype-genotype characterization. The phenotype is first characterized in surface-adhered cells. Then, the cells were fixed, and multiple rounds of hybridization were used to measure the barcodes. During the first round, readout probe 1-0 was added and cells with barcodes that read “0” in the first bit, which contained the readout sequence 1-0, should bind to the probe and become fluorescent, whereas cells with barcodes that read “1” in the first bit should remain dark. Once readout probe 1-0 was extinguished, readout probe 1-1 was added and the cells with barcodes that read “1” in the first bit, which contain the readout sequence 1-1, should become fluorescent. This difference in fluorescence intensity allows the value of bit 1 to be determined for each cell. This was repeated similarly for the remaining bits. After measuring all bits, the barcode was decoded, revealing the identity of each cell, the genotype of the genetic variant contained in the cell, and which phenotype the genotype corresponds to, FIG. 1C also demonstrates that the number of hybridization rounds can be reduced if multi-color imaging is utilized. Specifically, multiple readout probes, each conjugated to a different fluorophore, are hybridized simultaneously in one round, and multi-color imaging is used to probe the presence of each of these different readout probes.

### EXAMPLE 2

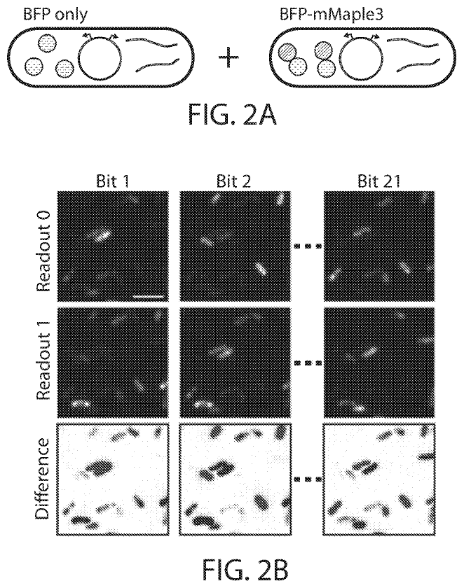

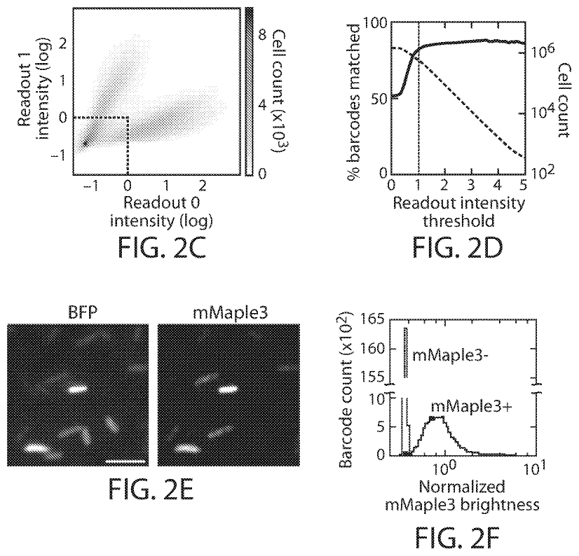

To test the accuracy of this screening approach, a library was created containing only two “genetic variants.” the mTagBFP2 gene and the fusion of mTagBFP2 and mMaple3 genes (FIG. 2A). Two libraries were created by merging a 21-bit barcode library, consisting of more than 2 million unique barcodes, with the two plasmids (containing the mTagBFP2 and mTagBFP2-mMaple3 genes, respectively) by isothermal assembly. Then, each of these complete libraries was bottlenecked to ˜40,000 unique members and sequenced to determine which of the ˜2 million possible barcodes are present in each library. Sequencing revealed that in the mixture of the two libraries, 80,000 unique barcodes are expected, which represent 4% of the possible barcodes.

This combined library was characterized using this screening strategy. The fluorescence properties of the cells expressing mTagBFP2 or mTagBFP2-mMaple3 were measured by illuminating with 405 nm light to measure mTagBFP2 fluorescence, illuminating with 405 nm light for an additional ˜4 s in order to switch the mMaple3 protein to its red-shifted fluorescent state, and then measuring the fluorescence intensity of the red-shifted mMaple3 by illuminating with 560 nm light. The cells were then fixed in methanol and barcodes were read out in each cell using the procedure described above. Indeed, as expected, cells that are bright for one readout of a given bit are dim for the other readout (FIG. 2B). For all 1.5 million cells observed, a two-dimensional (2D) histogram of the bit 1 measurements, i.e. the fluorescence intensities determined in the probe 1-0 imaging round and probe 1-1 imaging round, was constructed and this histogram suggested there were two distinct populations of cells (FIG. 2C). The first population appears bright when hybridized to probe 1-0 and dim when hybridized to probe 1-1 while the second population appears dim when hybridized to probe 1-0 and bright when hybridized to probe 1-1. This is consistent with the readout sequence 1-0 being present in the first population and readout sequence 1-1 being present in the second.

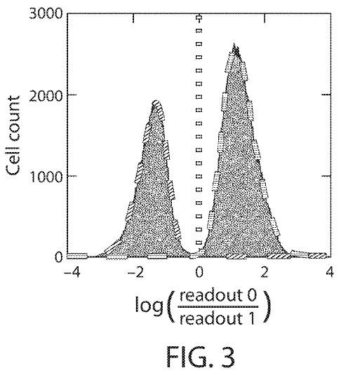

However, a substantial fraction of cells appeared dark in both imaging rounds, possibly because they were not expressing sufficient barcode RNA, or they were insufficiently permeabilized for readout probe hybridization. A threshold intensity was used to remove these cells from further analysis. Specifically, each set of intensities for each readout probe was normalized by the median value of all corresponding measurements and cells where both normalized readout intensities for the “0” and the “1” readout fell below 1 for any bit were removed. More than 600,000 measured cells were brighter than this intensity threshold and the barcodes expressed in these cells are determined. Among these cells, 84% of them matched a barcode contained in the library pre-determined by sequencing (FIG. 2D). Among cells assigned to valid barcodes, the distribution of “0”-to-“1” probe-intensity ratios for each bit showed two distinct cell populations with essentially zero overlap (FIG. 3).

For the unmatched 16% of the cells, an experimental error must have occurred. Either the barcode is present in the library but it was not detected by sequencing or the barcode is not present in the library and an error occurred during barcode imaging. Those cells were not used in further analysis. However, the presence of this unmatched fraction suggested that some of the barcodes in the 84% cells that match the barcodes in the library could also be mis-identified. In order to determine this misidentification rate, it was first noted that the library only contained 4% of all possible barcodes for the 21-bit binary encoding used, so assuming that a readout error in the imaging process is equally likely to result in a cell being assigned any of the 22 barcodes, there was a 96% chance that the error results in identifying a barcode that does not match one in the library.

Next, the probability that the barcode in a cell was incorrectly determined was denoted as x. Then, this probability x multiplied by 96% should be equal to 16%, the fraction of cells that were found containing barcodes that do not match any barcode in the library. Hence, x should be equal to 0.167 and the probability that the error results in a different barcode that is present in the library should be only 4% of x, which is ˜0.67%. Therefore, the estimated misidentification rate, i.e. the probability that both an error occurs and the error yields a barcode this is already present in the library, is only less than one percent.

The fidelity of the barcode measurement could also be verified by considering the phenotype measurements described above to determine the mTagBFP2 fluorescence intensity and the mMaple3 fluorescence intensity of each cell (FIG. 2E). All cells with identical barcode classifications were grouped and the median ratio of mMaple3 intensity to mTagBFP2 intensity was calculated for each. Since from sequencing, it was determined which barcodes should be associated with mMaple3, the median intensity ratio for these barcodes was calculated and a histogram was calculated with these ratios. Using the same approach, a histogram of the ratio for the barcodes known to only be associated with mTagBFP2 was calculated (FIG. 2F). These two distributions were largely separated with only small overlap. A threshold was set based on the intersection point of the two histograms such that the cells with fluorescence intensity ratio larger than this threshold was classified as containing the mTagBFP2-mMaple3 fusion protein and the cells with the intensity ratio below the threshold as containing mTagBFP2. Based on this criterion, it was found that less than 1% of cells had their barcodes misidentified. It was noted that this was an overestimate of the error, since not all cells crossed the threshold value into the other population is necessarily due to barcode mis-identification—the intensity spread of the fluorescent proteins could contribute to the crossing too.

FIG. 2 illustrates measuring 1.5 million cells containing 80,000 unique barcodes using a 21-bit code. FIG. 2A is a schematic diagram of the library constituents. Among the 80,000 distinct barcodes, half were associated with the mTagBFP2 gene while the other half were associated with the mTagBFP2 gene fused to the mMaple3 gene. The cells containing plasmids harboring these barcodes and the associated fluorescent protein genes would express both the barcode RNA and the fluorescent proteins. FIG. 2B shows fluorescent images for each readout of each bit. The difference of the two readout images for each bit determined the value of that bit in each cell. The first row displayed the readout 0 images of each bit (i.e. images with readout probes 1-0, 2-0, . . . , N-0) for a field of view and the second row displayed the readout 1 images (i.e. images with readout probes 1-1, 2-1, . . . , N-1). The third row displayed the difference images with the darker shading indicating that readout 0 intensity is greater than the readout 1 intensity, and the lighter shading indicate the opposite. Each bit was assigned either a “0” value or a “1” value based on the ratio of the readout 0 intensity to readout 1 intensity.

FIG. 2C shows a two-dimensional histogram of normalized fluorescence intensities for readout 0 and readout 1 of bit 1 for each cell. The fluorescence intensities were normalized to the median values. The dotted line depicted the threshold used for eliminating cells that appear dim in both readouts. FIG. 2D shows the percent of barcodes decoded in the imaging experiment that match barcodes determined to be in the library by sequencing (left) and number of cells above the bit readout intensity threshold (right, downward-sloping line) with varying threshold magnitude. The dotted line corresponded with the threshold of 1 shown in FIG. 2C. FIG. 2E shows a fluorescence image of BFP and fluorescence image of post-activation mMaple3 in the same region as FIG. 2B. FIG. 2F shows histograms of median mMaple3- fluorescence intensity normalized to mTagBFP intensity for barcodes associated with the mMaple3-mTagBFP2 fusion gene (rightward curve) and for those associated with the mTagBFP2-gene (leftward curve).

FIG. 3 shows a histogram of the natural logarithm of the ratio of readout 0 intensity to readout 1 intensity for bit 1 for only cells that were assigned barcodes that match barcodes that were determined to be in the library by sequencing. The histogram was fit to a sum of two skewed Gaussian curves and each fit Gaussian is depicted (dashed lines). The vertical dotted line depicts the bit-calling threshold.

### EXAMPLE 3

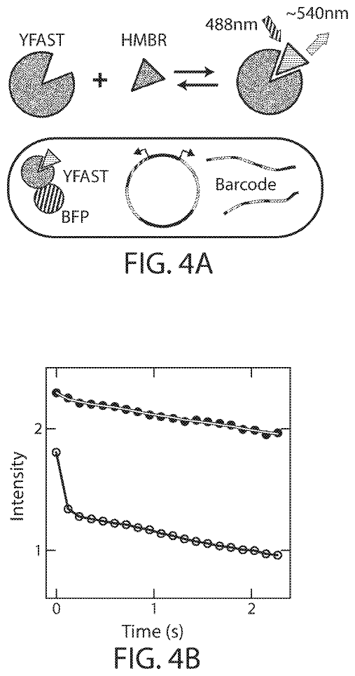

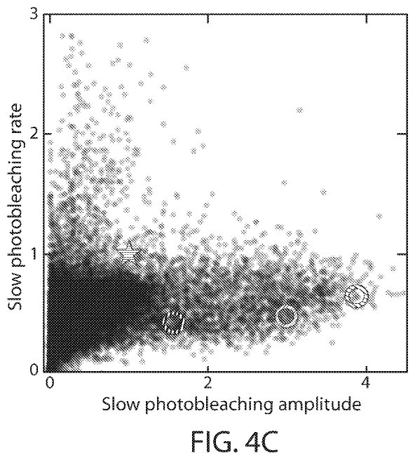

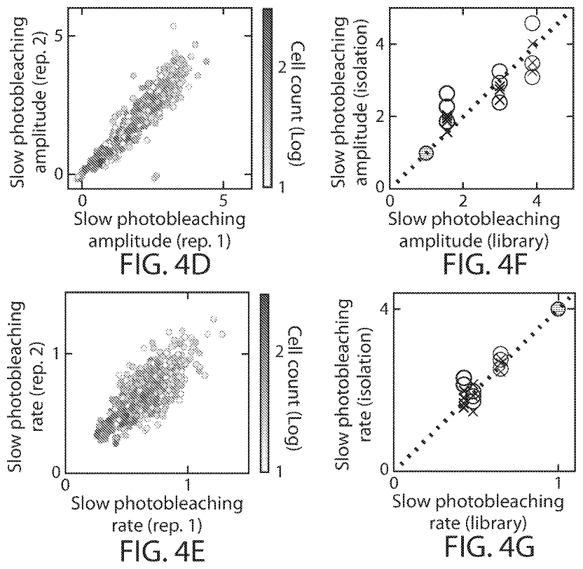

To demonstrate the utility of a high-throughput approach for screening a large library of mutants to find proteins with desired properties, this example screened for increased photostability and increased brightness of a recently developed fluorescent protein, YFAST. YFAST is a protein that is not itself fluorescent, but only becomes fluorescent upon binding to an exogenous. GFP-like chromophore known as HMBR (FIG. 4A). Libraries of YFAST variants were created that contained variants with single amino acid mutations throughout the whole protein in addition to variants with multiple mutations in residues in the vicinity of the chromophore. These libraries were assembled by incorporating synthetized DNA oligonucleotides designed to have sequences corresponding to the desired mutations into a purification plasmid. This plasmid filters frameshift and nonsense errors from the pool by using a downstream translational fusion to the chloramphenicol resistance gene. Then, the library of YFAST variants was merged with the 21-bit barcode library so that a single, unique barcode was randomly associated with each variant. To normalize for variation in expression levels of YFAST between different cells, YFAST was fused to mTagBFP2, a fluorescent protein that is spectrally distinct from YFAST. To determine which barcode was associated with each genetic variant, the barcoded genetic variant library was sequenced using high-throughput sequencing.

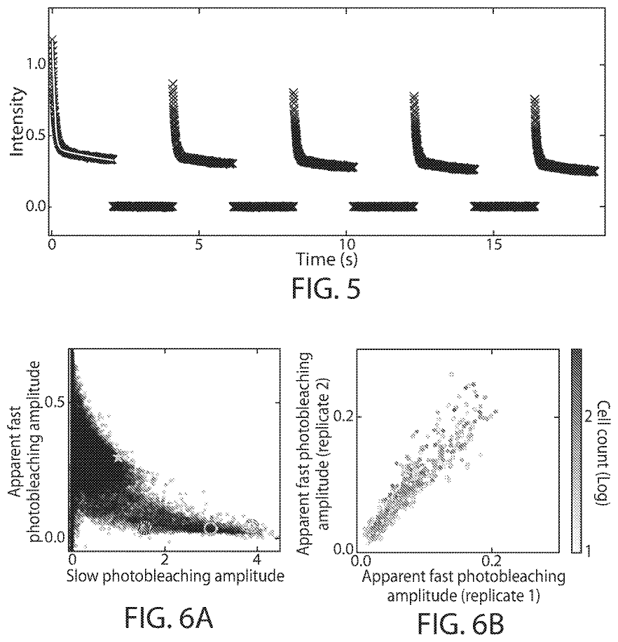

The library of barcoded YFAST variants was inserted into E. coli and the E. coli were adhered to a glass surface for imaging. First, the photophysical properties of the YFAST variant in each cell were measured. The fluorescence intensity of mTagBFP2 was measured in one camera image under 405 nm illumination. Then the photobleaching time-course of YFAST was measured over 20 images with constant 488 nm illumination (FIG. 4B). The acquired images were segmented to determine the boundaries of each cell and the average intensity for each cell was calculated for each image. The fluorescence background upon 488 nm illumination was subtracted from the photobleaching time series for each cell and each time series was normalized for cell-to-cell variation in expression levels of YFAST by dividing by the measured 405 fluorescence intensity. Since YFAST exhibits biphasic fluorescence decay upon photobleaching (FIG. 5), the background subtracted and normalized intensity time series for each cell was fit to a double exponential with the rate of the fast exponential fixed to a constant value (FIG. 4B and FIG. 5). From the fit, the slow photobleaching amplitude, slow photobleaching rate, and fractional fast photobleaching amplitude were determined for each cell.

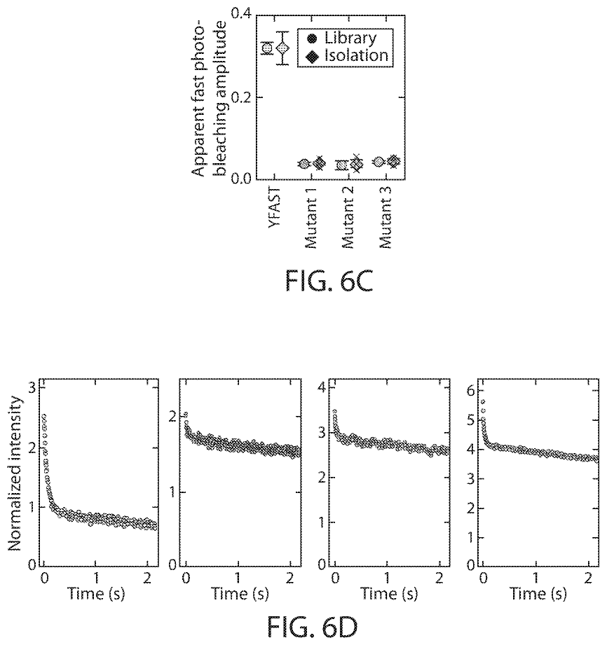

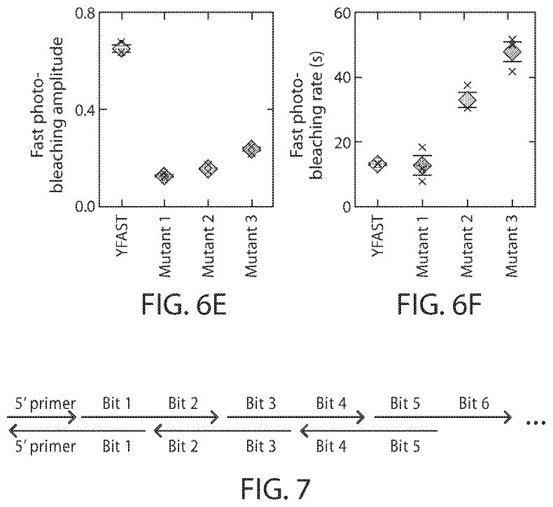

After measuring the phenotype of the YFAST variants, the cells were methanol fixed and the barcodes were measured as described above. The cells were grouped based on the measured YFAST mutant genotype and the median slow photobleaching amplitude, slow photobleaching rate, and relative fast photobleaching amplitude were calculated for each mutant (FIG. 4C and FIG. 6A). Altogether, ˜20 million cells containing ˜60,000 YFAST variants and ˜160,000 barcodes were screened. A subset of the library measurements was replicated and the measured parameters were reproduced (FIG. 4D, FIG. 4E, and FIG. 6B).

To further test the accuracy of the screen, three brighter and more photostable mutants were selected and characterized in homogeneous cultures where all bacteria express one of the selected mutants. As demonstrated by such measurements, these mutants were substantially brighter and more photostable than the original YFAST protein (FIG. 4F and FIG. 4G). Additionally, these isolated mutants were measured with higher temporal resolution to demonstrate that the magnitude and the rate of the fast photobleaching component are consistent with a decreased relative fast photobleaching amplitude measured in the screen with a lower temporal resolution (FIG. 6C, FIG. 6D, FIG. 6E, and FIG. 6F).

FIG. 4 shows screening YFAST mutant libraries for decreased slow-photobleaching rate and increased brightness. FIG. 4A shows a schematic diagram of YFAST library design. YFAST is dark on its own, but it becomes fluorescent upon binding to the ligand, HMBR. A library of YFAST variants fused to mTagBFP2 for normalization was merged with a library of barcodes and transformed into E. coli cells. FIG. 4B shows the phenotype measurement for two cells containing two variants of the YFAST protein. The fluorescence decay curve of the original YFAST (bottom curve) and a YFAST variant (top curve) was measured by illumination with 488 nm light to excite YFAST only. From each of these curves, the slow photobleaching rate, slow photobleaching amplitude, and fractional fast photobleaching rate were determined by fitting the curve with a double exponential decay. FIG. 4C shows a scatter plot of the slow photobleaching rate and the slow photobleaching amplitude for each mutant in the library. Library measurements of the original YFAST and three mutants are indicated by the star and open circles. FIG. 4D and FIG. 4E show the amplitudes (FIG. 4D) and rate constants (FIG. 4E) of the slow photobleaching component for two replicate measurements. Each filled circle in FIG. 4C-E depicts the median rate constants and amplitude of all cells associated with one mutant, and only mutants containing at least ten imaged cells are depicted. FIG. 4F and FIG. 4G show the amplitudes (FIG. 4F) and rate constants (FIG. 4G) of the slow photobleaching component of three selected mutants measured in isolation versus those measured in the library screen. Each point corresponds with a replicate of the isolation measurements conducted at the library-screen time resolution (120 ms; crosses) or at a 4-ms time resolution (circles). Amplitudes and rates are normalized to those of the original YFAST.

FIG. 5 shows the reversible and biphasic photobleaching kinetics of YFAST. The normalized fluorescence intensity (crosses) upon intermittent illumination with 488-nm light and a fit to a double exponential decay (solid line). E. coli expressing mTagBFP2-YFAST were adhered to a glass coverslip, immersed in 10 micromolar HMBR in PBS and imaged at 4-ms time resolution. The YFAST fluorescence intensity for each cell is normalized by the mTagBFP2 fluorescence and averaged over multiple cells in the imaged area. The 488-nm illumination was switched on and off with a period of 2 seconds. Intensity values of zero represent the period of time when the illumination was off.

FIG. 6 shows quantifications of the fast photobleaching amplitude from the same library measurements as FIG. 4. FIG. 6A depicts a scatter plot of apparent fast photobleaching amplitude and slow photobleaching amplitude for each mutant in the library. Library measurements of the original YFAST and three mutants are indicated by the star and open circles. FIG. 6B shows the apparent fast photobleaching amplitude for two replicate library measurements. Each filled circle in FIG. 6A and B depicts the amplitude of all cells associated with one mutant, and only mutants containing at least ten imaged cells are depicted. FIG. 6C shows the apparent fast photobleaching amplitude of three selected mutants from the library screen and their corresponding values measured in isolation with the same time resolution as the library measurements. FIG. 6D shows the fluorescence decay of the original YFAST and three select mutants measured at a 4-ms time resolution. The depicted mutants correspond to the open circles in FIG. 6A. FIG. 6E and F depict fractional photobleaching amplitude (FIG. 6E) and photobleaching rate (FIG. 6F) of the fast photobleaching component for the original YFAST and the selected mutants characterized in isolation measurements at a 4-ms time resolution.

In summary, these examples show methods for image-based screening of large genetic variant libraries by co-expressing the genetic variants and barcode that can identify these genetic variants in cells, and determining both the phenotypes of the genetic variants and the barcodes in the same cells using imaging. By reading out barcodes using massively multiplexed FISH, the ability to screen hundreds of thousands of barcodes that correspond to tens of thousands of unique genetic variations was shown. Using these techniques, mutations in the YFAST protein, a recently discovered ligand-dependent fluorescent protein with substantially improved brightness and photostability, were demonstrated in this example.

### EXAMPLE 4

Following are materials and methods used in some of the above examples.

Barcode library assembly. The barcode library used a set of plasmids, each containing a DNA barcode sequence that encodes a RNA designed to represent a single N-bit binary word. Every barcode in the library had N readout sequences, one corresponding to each bit, designed to be read out by hybridizing fluorescent probes with the complementary sequence. For each bit position, one 20-mer sequence was assigned to encode a value of “0” and another 20-mer sequence to encode a value of “1”. To increase the rate of hybridization, these encoding sequences were constructed from a three-letter nucleotide alphabet, one with only A, T, and C, in order to destabilize potential secondary structures. The utilized sequences were drawn from those previously used for MERFISH with additional sequences designed using approaches described previously. For each barcode, the bits were concatenated with a single G separating each. Although bits are present in the barcode set that was constructed here, to reduce the number of hybridization rounds, experiments were conducted by reading out either 21 or 18 of the possible bits, depending on the library size.

This barcode library was assembled by ligating a mixture of short, overlapping oligonucleotides, each representing a pair of adjacent bits (FIG. 7). For each pair of adjacent bits, there were four unique combinations of bit values (“00”, “01”, “10”, and “11”). Each corresponding sequence was synthesized as a single-stranded oligonucleotide. The oligonucleotides were then ligated to form complete, double-stranded barcodes that contain concatenated sequences of all bits with all possible bit values. For the ligation step, all oligonucelotides were mixed and diluted so that each oligo was present at a concentration of 100 nM. The mixture was phosphorylated by incubating with T4 polynucleotide kinase (16 microliter oligonucleotide mixture, 2 microliter T4 ligase buffer, 2 microliter PNK [NEB, M0201S]) at 37° C. for 30 minutes and ligated by adding 1 microliter T4 ligase (NEB, M0202S), and incubating for 1 hour at room temperature.

To prepare a plasmid library containing these barcode sequences under the control of the 1pp promoter, the ligation product was diluted 10-fold and amplified by limited-cycle PCR on a Bio-Rad CFX96 using Phusion polymerase (NEB. M0531SO) and EvaGreen (Biotium, 3100). The PCR product was run in an agarose gel, and the band of the expected length was extracted and purified (Zymo Zymoclean Gel DNA Recovery Kit, D4002). The purified product was inserted by isothermal assembly for 1 hour at 50° C. (NEB NEBuilder HiFi DNA Assembly Master Mix, E2621L) into a plasmid backbone fragment containing the colE1 origin, the ampicillin resistance gene, and other elements taken from the pZ series of plasmids. The assembled plasmids were purified (Zymo DNA Clean and Concentration, D4003), eluted into 6 microliter water, mixed with 10 microliter of electro-competent E. coli on ice (NEB, C2986K), and electroporated using an Amaxa Nucleofector II. Immediately after electroporation, 1 mL SOC was added and the culture was incubated at 37° C. on a shaker for one hour. Subsequently, the SOC culture was diluted into 50 mL of LB (Teknova, L8000) supplemented with 0.1 mg/mL carbenicillin (ThermoFisher, 10177-012) and placed on the shaker at 37° C. overnight. The following day, the culture was miniprepped (Zymo Zyppy Plasmid Miniprep Kit. D4019), yielding the complete barcode library.

Assembling protein mutant libraries. To create a library of mutant proteins, short nucleotide sequences containing regions of the protein with the desired mutations were synthesized as complex oligonucleotide pools. To then create the desired mutant genes from these pools, the pool and its corresponding expression plasmid was amplified via limited cycle PCR and these fragments assembled using isothermal assembly. The expression backbone was derived from the colE1 origin and the chloramphenicol resistance gene from the pZ series of plasmids. Oligonucelotide pool synthesis may be prone to deletions, which could lead to frameshift mutations that produce non-viable proteins. To remove these variants prior to measurement, the protein variants were translationally fused upstream to the chloramphenicol resistance protein. These constructs were electroporated into E. coli, as described above, and these cultures grown in the presence of chloramphenicol to select only for protein variants that did not have frame-shift mutations and which could, thus, translate competent chloramphenicol resistance. These plasmids were re-isolated via plasmid miniprep and the genetic variants extracted via PCR prior to combination with the barcode library.

Merging mutation libraries with the barcode library. To merge a mutant library with the barcode library, the corresponding halves of each plasmid library were amplified by limited-cycle PCR. Of note, the forward primer for amplifying the barcode library contained 20 random nucleotides so that each assembled plasmid contained a 20-mer unique molecular identifier (UMI). Also, the protein mutant half contained the plasmid's replication origin (colE1) while the barcode half contained the ampicillin resistance gene ensuring that only plasmids containing both halves were competent. The two halves were assembled by isothermal assembly and transfected into electrocompetent E. coli as described earlier. After incubating in SOC for 1 hour at 37° C., the culture was again diluted into 50 mL LB and grown until it reached an optical density at 600 nm (OD600) of ˜1. To limit the possibility that a single bacterium had taken up more than one plasmid, plasmids were extracted again from this culture and reinserted at a concentration where the number of E. coli cells significantly outnumbered the number of plasmids. Specifically, 2 microliter of the plasmid library at 100 pg/microliter was re-electroporated into 10 microliter of fresh electro-competent E. coli. This culture was then grown and diluted to a concentration of ˜1000 cells/microliter by using the OD600 to determine the number of cells in the culture and, thus, the appropriate dilution. From the diluted culture, a volume containing the desired number of cells, and hence the desired number of unique barcode-mutant pairs, was inoculated into a new culture. This culture was incubated at 37° C. overnight and the following day it was archived for future imaging experiments by diluting 1:1 in 50% glycerol (Teknova, G1796). separating into 100 microliter aliquots, and storing at −80° C. The remaining culture was mini-prepped to use as a PCR template for constructing the barcode to genotype lookup table.

Constructing the barcode-to-genotype lookup table. Since barcodes and gene variants were assembled randomly, next generation sequencing was used to construct a look-up table that links barcodes to their corresponding gene variant. The total length of the combined sequence of the gene variant and the barcode exceeded the read length of the sequencing platform used (Illumina MiSeq). To circumvent this challenge, multiple fragments were extracted from each library, sequenced independently and grouped computationally using the UMI.

The mini-prepped libraries were prepared for sequencing by two sequential limited-cycle PCRs. The first PCR extracted the desired region while adding the sequencing priming regions, and the second PCR added multiplexing indices and the Illumina adapter sequences. Between PCRs, the product was purified in an agarose gel and the final product was gel purified prior to sequencing.

For each sequencing read, the corresponding barcode or gene variant sequence was extracted. The reads were then grouped by common UMI, and the most frequently occurring barcode and gene variant seen for each UMI was assigned to that UMI, constructing the barcode-to-gene variant lookup table for every variant in the library. Any ambiguous barcode (i.e. a barcode assigned to more than one genetic variant) was excluded from further analysis. This analysis was conducted in custom software written in Matlab.

Library design of YFAST variants. Since YFAST is a recently developed fluorescent protein, the consequences of mutating different regions of the protein are not well characterized in the literature. Hence, the screen in this example was started by concurrently designing libraries following two distinct strategies. In the first strategy, a structurally naive view was used and a library was constructed (library type 1, LT1) having mutants corresponding to all possible single amino acid substitutions, insertions, and deletions at each location within YFAST. The second strategy made use of structural information of the YFAST precursor, Photoactive Yellow Protein (PYP) (PDB: 1NWZ) to target residues adjacent to the chromophore (library type 2, LT2-1), introducing up to 6 amino-acid substitutions per mutant. These libraries were screened using this screening method. Since many of the mutants in LT2-1 appeared dark, the selection of mutations was refined by redesigning the oligonucleotide pool to only include those amino-acid substitutions that appeared bright with relatively high frequencies in the LT2-1 library and another library (LT2-2) was created that combined these substitutions, containing up to 6 substitutions per mutant. This library was screened with this method as well. A library (library type 3, LT3) was created by combining mutations found to have favorable brightness and photostability (i.e. relatively large amplitude of the slow bleaching component) in LT1 with those mutations found to have favorable brightness and photostability in all LT2. Each variant in LT3 contains up to 10 mutations. LT3 was screened and a mutant with 6 amino acid substitutions that is particularly photostable with a large amplitude of the slow bleaching component and nearly eliminated the fast component at the library measurement time resolution was identified. Next, to further improve the fluorescent properties of this mutant, a new library (library type 4, LT4) that contained all possible single amino acid substitution, insertion, and deletion at every residue of this mutant was created. Finally, based on the screening results of LT4, library type 5 (LT5) was created by splitting the entire protein sequence into 6 regions, selecting LT4 mutations with favorable brightness and photostability in each region, and creating all possible combinations of these mutations. LT5 contains 6-12 mutations per library member.

Some of the above libraries were constructed and measured concurrently while developing and optimizing the screening protocol. Therefore, all of the libraries were re-measured again, by mixing them into pools containing ˜25,000 barcodes each. Instead of combining all libraries into a single pool and measuring a very large number of cells in a single screen over a long time, the measurements were split into smaller pools and measured at 1-2 million cells per experiment. Since the phenotype accuracy increases with the number of cells measured, the results from the earlier measurements of individual libraries that were performed using the optimized protocol were also included, FIG. 4 and FIG. 6 contain results from all library measurements performed with the optimized protocol.

Phenotype and barcode imaging. Each library was prepared for imaging by thawing the 100 microliter aliquot from −80° C. to room temperature and diluting into 2 mL LB supplemented with 0.1 mg/mL carbenicillin. Imaging coverslips (Bioptechs, 0420-0323-2) in 60-mm-diameter cell culture dishes were prepared by covering them in 1% polyethylenimine (Sigma-Aldrich, P3143-500ML) in water for 30 minutes followed by a single wash with phosphate buffered saline (PBS). The E. coli culture was diluted 10-fold into PBS, poured into the culture dish, and spun at 100 g for 5 minutes to adhere cells to the surface.

The sample coverslip was assembled into a Bioptech's FCS2 flow chamber. A peristaltic pump (Gilson, MINIPULS 3) pulled liquid through the chamber while three computer-controlled valves (Hamilton, MVP and HVXM 8-5) were used to select the input fluid. The sample was imaged on a custom microscope built around a Nikon Ti-U microscope body with a Nikon CFI Plan Apo Lambda 60× oil immersion objective with 1.4 NA. Illumination was provided at 405, 488, 560, 647, and 750 nm using solid-state single-mode lasers (Coherent, Obis 405 nm LX 200 mW; Coherent, Genesis MX488-1000; MPB Communications, 2RU-VFL-P-2000-560-BIR, MPB Communication, 2RU-VFL-P-1500-647-B1R; and MPB Communications, 2RU-VFL-P-500-750-B1R) in addition to the overhead halogen lamp for bright field illumination. The Gaussian profile from the lasers was transformed into a top-hat profile using a refractive beam shaper (Newport, GBS-AR14). The intensity of the 488-, 560-, and 647-nm lasers was controlled by an acousto-optic tunable-filter (AOTF), the 405-nm laser was modulated by a direct digital signal, and the 750-nm laser and overhead lamp were switched by mechanical shutters. The excitation illumination was separated from the emission using a custom dichroic (Chroma, zy405/488/561/647/752RP-UF1) and emission filter (Chroma, ZET405/488/461/647-656/752m). The emission was imaged onto an Andor iXon+888 EMCCD camera. During acquisition, the sample was translated using a motorized XY stage (Ludl, BioPrecision2) and kept in focus using a home-built autofocus system.

Phenotype measurements were conducted immediately after cells were deposited onto the coverslip, inserted into the flow chamber, and immersed in PBS. For imaging E. coli cells expressing mMaple3-mTagBFP2 fusion or mTagBFP2 alone, an image was first acquired for 1 frame with 405-nm illumination to excite mTagBFP2 at a frame rate of 8.4 Hz (120 ms), followed by illumination with 405-nm light for 30 additional frames at 8.4 Hz to photoactivate mMaple3. Then an image was acquired with 560-nm illumination for 1 frame to detect mMaple3 fluorescence. For imaging E. coli cells expressing the YFAST mutants, images were first acquired in the absence of the chromophore with 405-nm illumination for 1 frame to measure the mTagBFP2 fluorescence to determine the position of each cell followed by an image with bright-field illumination for alignment between multiple imaging rounds. Then 10 micromolar of the chromophore HMBR in PBS was flowed over the cells and a fluorescence image was acquired with 488-nm illumination for 1 frame to measure YFAST intensity, 405-nm illumination for 1 frame to measure mTagBFP2 intensity, and a bright-field image was acquired again for alignment, followed by at least 20 frames at 8.4 Hz with constant 488-nm illumination to measure the decrease in intensity upon photobleaching. Since 8.4 Hz is the full field frame rate of the camera that was used, increasing the time resolution would require imaging a smaller field of view per frame and hence a reduction in the measurement throughput. Images were acquired at thousands of locations in the sample, each corresponding to a ˜200×200 micrometer² field-of-view. All fields were imaged prior to the addition of the chromophore to determine the position of each cell, and then after the chromophore was added, all of the subsequent exposure sequence described above was completed at each field prior to moving to the next. The illumination intensities at the back-focal plane used in these experiments were 1 W/cm², 3 W/cm², and 10 W/cm² for the 405-nm, 488-nm, and 561-nm lasers, respectively. Following the phenotype measurement, the cells were fixed by incubation for 30 minutes in a mixture of methanol and acetone at a 4:1 ratio for fast hybridization to RNA. To prevent salts from precipitating and clogging the flow system, water was flowed before and after the fixation mixture. Once fixed, the cells were washed in 2×Saline Sodium Chloride (SSC) and hybridizations for MERFISH imaging were started.

To determine the RNA barcode expressed within each cell, multiple rounds of hybridizations were performed. For each hybridization round, the sample was incubated for 30 minutes in hybridization buffer (2×SSC; 5% w/v dextran sulfate (EMD Millipore, 3730-100 ML), 5% w/v ethylene carbonate (Sigma-Aldrich, E26258-500G). 0.05% w/v yeast tRNA, and 0.1% v/v Murine RNase inhibitor (NEB, M0314L)) with a mixture of readout probes labeled with either ATTO565, Cy5, or Alexa750 (Bio-Synthesis Inc.) each at a concentration of 10 nM). In the readout probes, the dyes were linked to the oligonucleotides through a disulfide bond. Then, the hybridization buffer was replaced by an oxygen-scavenging buffer for imaging (2×SSC; 50 mM TrisHCl pH 8. 10% w/v glucose (Sigma-Aldrich, G8270), 2 mM Trolox (Sigma-Aldrich, 238813), 0.5 mg/mL glucose oxidase (Sigma-Aldrich, G2133), and 40 μg/mL catalase (Sigma-Aldrich, C100-500 mg)). Each position in the flow cell was imaged with 750-, 647-, and 560-nm illumination from longest to shortest wavelength followed by bright-field illumination for alignment before continuing to the next location. Following the imaging of all regions, the disulfide bonds linking the dyes to the oligonucleotides in the readout probes were cleaved by incubating the sample in 50 mM tris (2-carboxyethyl)phosphine (TCEP; Sigma-Aldrich, 646547-10X1ML) in 2×SSC for 15 minutes. The sample was then rinsed in 2×SSC and the next hybridization round started. For each round of hybridization, three readout probes with spectrally discernable dyes (ATTO565, Cy5, and Alexa750) were hybridized simultaneously as described above. Altogether, with 14 hybridization rounds, all 42 readouts corresponding to 21 bits were measured in 40 hours. For smaller libraries, the imaging area was reduced, and the number of hybridization rounds was decreased to 12 (for 18-bit readout), reducing the measurement time to 22 hours.

Image analysis. To correct for residual illumination variations across the camera, a flat-field correction was performed as follows. Every image was divided by the mean intensity image for all images with the given illumination color. Then, the images for different rounds corresponding to the same region were aligned using the image acquired under bright field illumination by up-sampled cross-correlation, creating a normalized image stack of all images at each position in the flow chamber. If the radial power spectral density of any given bright field image did not contain sufficient high frequency power, the image was designated as out-of-focus and all images for the corresponding region were excluded from further analysis.

To extract cell intensities, the edges of each cell were detected using the Canny edge detection algorithm on the image acquired with 405-nm illumination for mTagBFP2 imaging. The edges that formed closed boundaries were filled in and closed regions of pixels were extracted. If a given closed pixel region had a filled area of more than 20 pixels and the ratio of the filled area to the area of the convex hull was greater than 0.9, it was classified as a cell. To increase the cell detection efficiency, the detected cells were then removed from the binary image, the image was dilated, filled, and eroded and cells were extracted again. This allowed cells where gaps exist in the detected edges to still be detected. For each cell, the mean intensity was extracted for the corresponding pixels in every image.

From the cell intensities, the phenotypes and barcodes were calculated. For each measured readout sequence, the measured intensity was normalized by subtracting the minimum and dividing by the median signal observed for that readout sequence across all cells. To determine whether a barcode contained a “1” or a “0” at each bit, the measured intensities of the “1” readout sequence and the “0” readout sequence for that bit were compared. Specifically, a threshold was selected on the ratio of these two values, called the “0”-to-“1” intensity ratio. If the “0”-to-“1” intensity ratio was above the threshold, the bit was called as a “0”. Otherwise, the bit was called as a “1”. Because the “1” and “0” readout sequences were measured in different hybridization rounds and there was variation in staining quality between rounds, this threshold was optimized for each bit individually. This optimization was performed by randomly selecting 150 barcodes (a training set) from the set of known barcodes that were determined to be present in the library by sequencing. An initial set of thresholds was selected and the fraction of cells matching these barcodes was determined. The threshold for each bit was then varied independently to identify the threshold set that maximizes this fraction. This optimized threshold set was then used for determining the bit values for all cells.

Once the barcode was determined for each cell, cells were grouped by barcode and the median of the various phenotype values was computed to determine the measured phenotype for the genotype corresponding to that barcode. For the mMaple3 measurement, the normalized brightness was determined from the ratio of the mMaple3 intensity under 560-nm illumination to the mTagBFP2 intensity under 405-nm illumination, as discussed above. For YFAST measurements, the normalized intensity was determined by the ratio of the YFAST fluorescence intensities under 488-nm illumination in the presence of the YFAST chromophore HMBR to the mTagBFP2 fluorescence intensities under 405-nm illumination. To account for the fluorescence background present in E. coli upon 488-nm illumination, the background was independently determined and subtracted before calculating the fluorescence ratio. The background was estimated by calculating the median intensity of all cells upon 488-nm illumination predicted to contain a non-fluorescent YFAST mutant. Specifically, cells, grouped by barcode, were assigned to the non-fluorescent population if the Pearson correlation coefficient between the fluorescence intensity measured under 488-nm illumination (YFAST channel) and those measured under 405-nm illumination (mTagBFP2 channel) for the grouped cells fell below a threshold of 0.2. Since the YFAST variant is translationally fused to mTagBFP2, when the two intensities are uncorrelated, it suggests that the number of YFAST proteins in the cells does not affect the brightness of the cell and hence the YFAST associated with that barcode should be dark.

The initial high-time resolution (4-ms) measurements of the original YFAST variant revealed a biphasic decay of fluorescence with time. To quantify this behavior, the background-subtracted photobleaching curve, b(t), was fit to the sum of two exponentials:

b(t)=P_{fast}e^{-At}+p_{slow}e^{-Bt}

where P_{fast} and A represent the amplitude and decay rate constant for the fast photobleaching component and P_{slow} and B represent the corresponding values for the slow photobleaching component. These fits of the original YFAST showed that the decay rate constants for the fast and slow components were ˜10 s−1 and ˜0.1 s−1, respectively, under this illumination intensity.

This double-exponential decay function was also used to characterize the library screen measurements. However, to increase the throughput of the screens, the full imaging frame of the camera was utilized, which required the use of a slower frame rate (8.4 Hz, ˜120 ms). This frame rate was comparable to the decay rate observed for the fast component of the original YFAST variant; thus, it was not anticipated that the rate constant associated with the fast component would be well constrained by this double-exponential fit. To address this, the rate constant of the fast component was initially fixed to the value determined from the original YFAST and the other three parameters allowed to vary in the fit. The time resolution of the library measurements was much higher than the decay time constant of the slow component; thus, the parameters associated with the slow component. P_{slow} and B, were well constrained by this fit—a point confirmed by the observation that P_{slow} and B did not change appreciably (by <0.5%) when the fixed value of A was varied over a wide range, or A was also used as a fitting parameter. Furthermore, it was anticipated that the time resolution, 120 ms, and duration, 2.5 s, of the library measurements, plus the independent determination of the background level (discussed above), should allow P_{slow} and B to be determined reliably. Though there was a possibility that beyond the measurement duration, YFAST displays more complicated photobleaching kinetics with more decay rate constants, in which case, the reported rate constant B for the slow component should be considered the initial decay rate of this component. To estimate the fast component amplitude, P_{fast}, the well-constrained value of the slow component amplitude, P_{slow}, was utilized. Specifically, P_{fast} was calculated from the difference of the initial brightness of each variant (P_{fast}+P_{slow}) and the fit value for the slow component amplitude, P_{slow}. Because of the limited time resolution of the library screen, the rate constant of the fast bleaching component was not extracted. It was noted that the apparent amplitude that was determined for the fast bleaching component may systematically underestimate this amplitude. Nonetheless, it should still provide useful information for future imaging experiments using the YFAST variants at ˜100 ms or slower time resolution.

The reported values for the slow component amplitude and decay rate were normalized to the corresponding values measured for the original YFAST, unless otherwise mentioned. The fast photobleaching component amplitude was not normalized in this fashion but rather was reported as the fraction of the total brightness, which was termed the fractional fast photobleaching amplitude.

This analysis was conducted in custom software written in Python.

### EXAMPLE 5

Considerations when designing a high-throughput screen. There are several aspects that should be taken into account when designing a high-throughput screen, including, for example, the number of bits in the barcodes, the fraction of possible barcodes used, and the number of cells that should be measured per variant to allow phenotypes to be measured accurately. This example summarizes some points that may be considered when designing a screen to measure the phenotypic variability within a given library.

Bottlenecking barcodes. In these examples, only a small fraction of all possible N-bit binary barcodes were used in a library, and this bottlenecking strategy served two purposes: (i) to limit the frequency with which the same barcode might be associated with two or more different genetic variants and (ii) to introduce an error robustness into this barcode-to-genotype identification process. In the construction of the barcoded genetic variants, barcodes were associated with individual genetic variants randomly, hence the probability that a given barcode could be assigned to multiple different genetic variant could be high. While this situation may be detected via next-generation sequencing when the barcode-to-genetic variant lookup table is built, these barcodes may also need to be discarded from the library screen measurement since cells containing such barcodes could not be unambiguously assigned to a given genotype. If a large fraction of the used barcodes were associated with multiple genetic variants, the number of barcodes that would need to be discarded would be high. To overcome this, the number of barcodes used in the library discussed above was restricted to be less than 10% of the total number of possible N-bit binary barcodes. Specifically, after the barcoded genetic variants were assembled, the size of the barcoded genetic variants library was bottlenecked such that the number of genetic variant-barcode pairs in the library was <10% of the total number of possible N-bit binary barcodes. Because only such a small fraction of barcodes are included, most barcodes would be present only once in the library, and the chance that a barcode was present more than once (hence allowing the possibility of being paired with more than one genetic variant) was very small (<10%). The remaining small fraction of barcodes that were paired with more than one variant could be detected by sequencing and discarded in further analysis.

The second reason why bottlenecking was used was to introduce error robustness into the genotype identification process. Specifically, if only a relatively small fraction of all possible barcodes was used, barcode measurement errors would more likely produce a barcode that is not present in the library, i.e. an invalid barcode. Because the exact barcodes that are present in the library via next-generation sequencing are known, it is possible to identify the invalid barcodes that resulted from errors during barcode imaging and discard them. This ability greatly reduced the rate at which the genotype of a given cell was misidentified. For example, if the barcode number was bottlenecked such that <10% of the total possible barcodes are present in the library, the chance that a barcode imaging error would lead to genotype misidentification will be reduced to <10%.

In the above experiments, a degree of bottlenecking was chosen such that only 1-10% of the possible 21-bit binary barcodes was present in the libraries. The bottlenecking was achieved experimentally by selecting a small. random subset of cells after transforming E. coli cells with the barcode-mutant plasmids under the condition that each cell contains a unique barcode-mutant pair. For example, to achieve a bottlenecking degree of 4%, the number of cells that is 4% of the number of possible 21-bit binary barcodes was selected.

Determining the number bits in the barcodes. The number of bits in the barcode was determined by the number of gene variants that was needed to screen. While optimizing YFAST, mutant libraries were created in two ways: (1) The first type of libraries contained a defined, relatively small number of mutants that was hoped to be screened exhaustively; (2) the second type of libraries contained a very large number of possible mutants where screening only a random subset of these mutants would already be very informative. When the first type of libraries was created, a barcode diversity was chosen such that the number of barcodes in the library was 5 times more than the number of unique mutants to ensure that each mutant (or at least the vast majority of them) was present in the library at least once. Because of the bottlenecking strategy describe above, namely the number of barcodes in the library being <10% of the total number of possible N-bit binary barcodes, the total number of possible barcodes needed to be 50 times more than the number of mutants to screen. Based on this number, the desired number of bits was determined. For example, if 20,000 specific mutants needed to be screened, more than 1 million possible barcodes would be needed, and hence a 21-bit barcoding scheme that can give ˜2 million possible barcodes was used. When the second type of libraries was created in which only a subset of possible mutants will be screened, a library size was selected to be equal to the number of mutants that was intended to subsample from the larger library; in this case, each mutant in the library was only associated with a single barcode and the number of barcodes in the library was equal to the number of mutants to be screened. The number of possible N-bit binary barcodes and hence the number of bits required were then likewise determined based on the bottlenecking strategy.

Determining the desired number of measured cells per genetic variant. In the library screens, the number of cells that need to be measured for each genetic variant was largely determined by the accuracy of the phenotype measurement. As the number of cells measured for each genotype increases, the accuracy with which that phenotype is measured improves. The desired cell number per genetic variant was set by the noise properties of the screened phenotype and the measurement accuracy that is needed to discriminate phenotype variations.

For the screen of YFAST variants, a large cell-to-cell variance in the fluorescence intensity measurements was observed between cells expressing the same genotype. This variance was observed even within a monoculture of the original YFAST. This observation indicated that the measurement accuracy of this type of phenotype from a single cell was low and, thus, required screening many more cells than mutants to increase this accuracy. In addition, it was found that different mutants appear in different abundance within the libraries, and this natural variation arose because of the random processes of constructing the plasmid-mutant libraries and transforming E. coli. To ensure that the majority of mutants were measured with a desired number of cells, this abundance variation further increased the oversampling requirement. For the YFAST measurements, ˜100 cells on average per mutant were measured.

Finally, in the genotype (barcode) measurements, a substantial fraction of the cells were discarded by readout intensity thresholding and by the rejection of barcodes that do not match the valid barcodes present in the library, as described above. In the above measurements, ˜66% of the measured cells were discarded because of the above procedures. As a result, on average, 300 cells per YFAST variant needed to be measured to achieve of the goal of ˜100 cells per mutant. Therefore, 20 million cells were measured to screen 60,000 YFAST variants in these experiments.

Since the noise properties of the screened phenotype and the measurement accuracy that was needed to discriminate phenotype variations both depend on the phenotype to be screened, the number of cells that needs to be measured per genotype depended on the phenotype to be screened. It is worth noting that given the reproducibility between phenotypes measured for the same genotype in separate screens, it may also be possible to increase the number of cells measured on average for a given library by simply replicating the screen multiple times with the same library and pooling the results so as to improve the accuracy of phenotype variability if it is determined not to be sufficient from a single measurement, in certain embodiments.

Estimate of the maximum plausible library size of the genetic variants. There are multiple factors that determine the maximum library size of genetic variants that can be screened. The first potential limitation to the size of the library is the number of unique barcodes that can be measured. These experiments demonstrated the ability to image 21-bit barcodes, and degradation in the image quality between the last imaged bit and the first imaged bit was not observed. Thus, adding more bits to the barcode should be possible. For example, 25-bit barcodes can also readily be measurable. Moreover, given such a modest extension in the length of the barcode, constructing plasmids that contain 25-bit barcodes (or other barcodes with 22 or more bits), or creating a barcode-mutant lookup table using existing next-generation sequencing approaches (Illumina HiSeq or NovaSeq). As a non-limiting example, 25-bits would produce ˜30 million possible barcodes. Based on the bottlenecking strategy, <10% of the possible barcodes can be selected to include in the library, which means <3 million barcodes to include in the library. By utilizing high-competency E. coli strains, as shown here, 10-fold more transformants than library members can be created, a sufficient coverage level, by pooling a few transformation reactions. If the aim is to see each mutant (or the vast majority of them) at least once, 5 times more barcodes in the library than the number of genetic variants can be used, which, in this case, means the library could contain up to ˜600.000 genetic variants. Assuming that 10-100 cells per variant were measured on average (depending on the phenotype measurement accuracy requirement), and based on the current settings of the barcode readout intensity threshold, in which ⅓ of cells pass the threshold and generate correct barcodes, ˜18-180 million cells should be measured. In the measurements demonstrated here, ˜1-2 million E. coli cells were characterized in a 40-hour long screen. However, in these measurements a relatively low density of E. coli on the coverslips were used, so as to minimize the chance of cells contacting each other. This density could be increased by at least 10-fold, e.g., without producing substantial cell-cell contact, and improvement in cell-segmentation algorithms should also allow contacting cells to be properly segmented. Thus, the density of cells is not a critical factor. Accordingly, measuring ˜18-180 million cells with a reasonable imaging time (e.g., 2-18 days) is reasonable.

Moreover, there are multiple ways that the protocols could be modified so as to further increase throughput. For example, improved hybridization approaches can reduce the number of dim or dark cells, allowing more of the measured cells to be utilized in the screen. Lower magnification objectives can be used to measure much larger fields of view and hence allow substantial improvements in the measurement throughput. Low magnification for genotype (barcode) imaging while keeping the use of high magnification for the high-resolution phenotype measurements can be used in some embodiments, because the phenotype measurements are typically fast and the total imaging time of the screen is dominated by barcode imaging which requires many rounds of hybridization. In addition, the number of barcodes can be increased in some embodiments, for example, by either increasing the number of bits in the binary barcode scheme, and/or by using higher order barcoding schemes, such as ternary or quaternary schemes, etc.

### EXAMPLE 6

Following are some of the sequences used in the above examples.

Original YFAST, amino acid sequence:
(SEQ ID NO: 1)
EHVAFGSEDIENTLAKMDDGQLDGLAFGAIQLDGDGNILQYNAAEGDITG
RDPKQVIGKNFFKDVAPGTDSPEFYGKFKEGVASGNLNTMFEWMPTSRGP
TKVKVHMKKALSGDSYWVFVKRV
Original YFAST, nucleotide sequence:
(SEQ ID NO: 2)
GAACATGTGGCGTTTGGAAGTGAGGACATTGAGAATACGCTTGCGAAGAT
GGATGATGGTCAACTGGATGGTCTTGCCTTTGGAGCAATTCAGTTGGATG
GCGATGGTAACATCTTGCAGTACAATGCCGCCGAGGGTGATATTACAGGA
CGTGATCCCAAACAAGTGATTGGAAAAAATTTTTTCAAAGATGTAGCGCC
TGGCACTGACTCACCCGAGTTTTACGGTAAGTTCAAAGAAGGCGTGGCTT
CCGGTAATCTTAATACGATGTTTGAGTGGATGATCCCCACTAGCCGTGGA
CCCACCAAGGTGAAAGTGCATATGAAGAAGGCCTTATCGGGCGATAGCTA
CTGGGTGTTCGTTAAACGTGTT
Example improved YFAST, amino acid sequence:
(SEQ ID NO: 3)
EHVAFGSEDIENTLAKMDDGQLDGLAFGAIQLDGDGNILQYNAAEGDITG
RDPKQVIGKNLFKDVACGTRSSEFYGKFKEGVASGNLNTMFEWMIPTSRG
PTKVKVHMKKALSGDSYWVFVKRV
Example improved YFAST, nucleotide sequence:
(SEQ ID NO: 4)
GAACATGTGGCGTTTGGAAGTGAGGACATTGAGAATACGCTTGCGAAGAT
GGATGATGGTCAACTGGATGGTCTTGCCTTTGGAGCAATTCAGTTGGATG
GCGATGGTAACATCTTGCAGTACAATGCCGCCGAGGGTGATATTACAGGA
CGTGATCCCAAACAAGTGATTGGAAAAAATCTGTTCAAAGATGTAGCGTG
CGGCACTCGTTCAAGCGAGTTTTACGGTAAGTTCAAAGAAGGCGTGGCTT
CCGGTAATCTTAATACGATGTTTGAGTGGATGATCCCCACTAGCCGTGGA
CCCACCAAGGTGAAAGTGCATATGAAGAAGGCCTTATCGGGCGATAGCTA
CTGGGTGTTCGTTAAACGTGTT
mTagBFP2, amino acid sequence:
(SEQ ID NO: 5)
MVSKGEELIKENMHMKLYMEGTVDNHHFKCTSEGEGKPYEGTQTMRIKVV
EGGPLPFAFDILATSFLYGSKTFINHTQGIPDFFKQSFPEGFTWERVTTY
EDGGVLTATQDTSLQDGCLIYNVKIRGVNFTSNGPVMQKKTLGWEAFTET
LYPADGGLEGRNDMALKLVGGSHLIANAKTTYRSKKPAKNLKMPGVYYVD
YRLERIKEANNETYVEQHEVAVARYCDLPSKLGHKLN
mTagBFP2, nucleotide sequence:
(SEQ ID NO: 6)
ATGGTGTCTAAAGGAGAGGAACTTATCAAAGAAAATATGCACATGAAGCT
TTACATGGAAGGAACAGTGGACAATCACCATTTTAAATGTACATCAGAAG
GCGAGGGTAAACCTTATGAGGGGACGCAAACCATGCGTATCAAGGTCGTA
GAGGGCGGCCCTTTGCCTTTCGCTTTCGACATTCTTGCAACCTCATTCTT
GTATGGCTCCAAGACTTTTATCAACCATACACAAGGCATTCCCGATTTCT
TTAAACAATCGTTCCCTGAAGGTTTTACATGGGAACGTGTAACAACATAT
GAAGATGGGGGAGTTTTAACTGCCACACAGGATACATCTTTACAGGATGG
CTGCCTGATCTATAATGTAAAGATCCGTGGAGTGAACTTTACCTCGAACG
GCCCCGTCATGCAGAAAAAGACCCTTGGGTGGGAGGCCTTTACGGAAACG
CTTTACCCCGCGGACGGAGGTCTGGAAGGACGTAATGACATGGCGCTGAA
GCTTGTCGGAGGATCCCATCTGATCGCAAATGCTAAGACCACCTATCGTA
GCAAGAAACCTGCTAAAAACTTAAAAATGCCTGGTGTTTACTACGTGGAC
TATCGTCTTGAGCGTATTAAGGAGGCAAATAACGAAACCTATGTTGAACA
ACACGAGGTCGCTGTGGCCCGCTATTGCGACTTGCCCTCGAAGCTGGGGC
ATAAGTTGAAT
mMaple3, amino acid sequence:
(SEQ ID NO: 7)
VSKGEETIMSVIKPDMKIKLRMEGNVNGHAFVIEGEGSGKPFEGIQTIDL
EVKEGAPLPFAYDILTTAFHYGNRVFTKYPRKIPDYFKQSFPEGYSWERS
MTYEDGGICNATNDITMEEDSFINKIHFKGTNFPPNGPVMQKRTVGWEVS
TEKMYVRDGVLKGDVKMKLLLKGGSHYRCDFRTTYKVKQKAVKLPKAHFV
DHRIEILSHDKDYNKVKLYEHAVARNSTDSMDELYK
mMaple3, nucleotide sequence:
(SEQ ID NO: 8)
GTTAGCAAGGGCGAGGAGACCATCATGAGCGTGATCAAGCCGGACATGAA
GATCAAGCTGCGCATGGAGGGCAACGTGAACGGCCATGCCTTTGTGATCG
AGGGCGAGGGCAGCGGTAAGCCGTTTGAGGGCATCCAGACCATCGACCTG
GAGGTTAAGGAAGGCGCACCGCTGCCGTTTGCCTACGACATCCTGACCAC
CGCATTCCACTACGGCAACCGCGTGTTCACCAAGTACCCGCGCAAGATCC
CGGACTACTTCAAGCAGAGCTTCCCGGAGGGCTACAGTTGGGAACGCAGC
ATGACCTACGAGGACGGCGGTATCTGCAACGCCACCAACGACATCACCAT
GGAAGAAGATAGCTTCATCAACAAGATCCACTTCAAGGGCACAAACTTCC
CGCCGAATGGTCCGGTTATGCAGAAGCGCACCGTTGGCTGGGAGGTGAGC
ACCGAGAAGATGTATGTGCGCGACGGTGTGCTGAAGGGCGACGTGAAGAT
GAAGCTGCTGCTGAAGGGTGGCAGCCACTACCGCTGCGACTTCCGCACCA
CCTACAAAGTTAAGCAAAAGGCAGTGAAGTTACCGAAGGCCCACTTCGTG
GACCACCGCATCGAAATCCTGAGCCACGACAAGGACTATAACAAAGTGAA
GCTGTACGAGCACGCCGTGGCCCGTAACAGCACCGACAGCATGGATGAGC
TGTACAAA

Below explains a barcode library used in some of the above examples. The barcodes are 22 bits but only 21 of the bits were read out in the experiments described above. Each barcode has 22 readout sequences, one sequence corresponding to each bit. The 22 bit sequences used in the barcode library were:

Readout 1-1:
(SEQ ID NO: 9)
ATCCTCCTTCAATACATCCC
Readout 1-0:
(SEQ ID NO: 10)
TATCTCATCAATCCCACACT
Readout 2-1:
(SEQ ID NO: 11)
ACACTACCACCATTTCCTAT
Readout 2-0:
(SEQ ID NO: 12)
AAACACACACTAAACCACCC
Readout 3-1:
(SEQ ID NO: 13)
ACTCCACTACTACTCACTCT
Readout 3-0:
(SEQ ID NO: 14)
AACTCATCTCAATCCTCCCA
Readout 4-1:
(SEQ ID NO: 15)
ACCCTCTAACTTCCATCACA
Readout 4-0:
(SEQ ID NO: 16)
AATACTCTCCCACCTCAACT
Readout 5-1:
(SEQ ID NO: 17)
ACCACAACCCATTCCTTTCA
Readout 5-0:
(SEQ ID NO: 18)
TCTATCATCTCCAAACCACA
Readout 6-1:
(SEQ ID NO: 19)
TTTCTACCACTAATCAACCC
Readout 6-0:
(SEQ ID NO: 20)
TCCAACTCATCTCTAATCTC
Readout 7-1:
(SEQ ID NO: 21)
ACCCTTTACAAACACACCCT
Readout 7-0:
(SEQ ID NO: 22)
TTCCTAACAAATCACATCCC
Readout 8-1:
(SEQ ID NO: 23)
TCCTATTCTCAACCTAACCT
Readout 8-0:
(SEQ ID NO: 24)
ATAAATCATTCCCACTACCC
Readout 9-1:
(SEQ ID NO: 25)
TATCCTTCAATCCCTCCACA
Readout 9-0:
(SEQ ID NO: 26)
ACCCAACACTCATAACATCC
Readout 10-1:
(SEQ ID NO: 27)
ACATTACACCTCATTCTCCC
Readout 10-0:
(SEQ ID NO: 28)
TACTACAAACCCATAATCCC
Readout 11-1:
(SEQ ID NO: 29)
TTTACTCCCTACACCTCCAA
Readout 11-0:
(SEQ ID NO: 30)
ACTTTCCACATACTATCCCA
Readout 12-1:
(SEQ ID NO: 31)
TTCTCCCTCTATCAACTCTA
Readout 12-0:
(SEQ ID NO: 32)
TTCTTCCCTCAATCTTCATC
Readout 13-1:
(SEQ ID NO: 33)
ACCCTTACTACTACATCATC
Readout 13-0:
(SEQ ID NO: 34)
AATCTCACCTTCCACTTCAC
Readout 14-1:
(SEQ ID NO: 35)
TCCTAACAACCAACTACTCC
Readout 14-0:
(SEQ ID NO: 36)
ACCTTTCTCCATACCCAACT
Readout 15-1:
(SEQ ID NO: 37)
TCTATCATTACCCTCCTCCT
Readout 15-0:
(SEQ ID NO: 38)
TCCTCATCTTACTCCCTCTA
Readout 16-1:
(SEQ ID NO: 39)
TATTCACCTTACAAACCCTC
Readout 16-0:
(SEQ ID NO: 40)
TCAAACTTTCCAACCACCTC
Readout 17-1:
(SEQ ID NO: 41)
TTACCTCTAACCCTCCATTC
Readout 17-0:
(SEQ ID NO: 42)
ACACCATTTATCCACTCCTC
Readout 18-1:
(SEQ ID NO: 43)
TCCCAACTAACCTAACATTC
Readout 18-0:
(SEQ ID NO: 44)
ACATCCTAACTACAACCTTC
Readout 19-1:
(SEQ ID NO: 45)
ATCCTCACTACATCATCCAC
Readout 19-0:
(SEQ ID NO: 46)
TCTCACACCACTTTCCTCAT
Readout 20-1:
(SEQ ID NO: 47)
TCCCTATCAATCTCCATAAC
Readout 20-0:
(SEQ ID NO: 48)
TTATCCATCCCTCTTCCTAC
Readout 21-1:
(SEQ ID NO: 49)
TCACCTCTAACTCATTACCT
Readout 21-0:
(SEQ ID NO: 50)
TCCTACAACATCCTTCCTAA
Readout 22-1:
(SEQ ID NO: 51)
ATCTCCCTTCTCTTCTCATA
Readout 22-0:
(SEQ ID NO: 52)
ATTACACCTCAACCCACACA

The various readout bits (Readout 1, Readout 2, Readout 3, . . . Readout 22) were sequentially combined to produce the final sequence. So, for example, one barcode might have a structure:

Readout 1-1- Readout 2-0- Readout 3-0- . . . -Readout 22-1

and it would have the sequence:

(SEQ ID NO: 53)
ATCCTCCTTCAATACATCCC AAACACACACTAAACCACCCAACTCATCT
CAATCCTCCCA . . . ATCTCCCTTCTCTTCTCATA

and encode the binary word:

100 . . . 1

All possible combinations of the 0 readout and the 1 readout for each bit (2²² combinations, or over 4 million sequences) were used in the barcode library.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list. “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-201. (canceled)

202. A composition for correlating a genotype and a phenotype of a genetic variant in a population of cell, comprising:
a pool of nucleic acid constructs, wherein each construct comprises a sequence encoding a distinct variant operably linked to a barcode sequence encoding an N-bit binary barcode assigned to the distinct variant, wherein the barcode sequence comprises a pair of read sequences for each N position of the binary code wherein one of the read sequences of the pair is assigned to encode a value of “0” and the other read sequence of the pair is assigned to encode a value of “1”.

203. The composition of claim 202, wherein the nucleic acid construct encodes at least two distinct variants.

204. The composition of claim 202, wherein the barcode sequence encodes at least a 3-bit binary barcode and wherein each possible combination of the N-bit binary barcode is present within the pool of nucleic acid constructs.

205. The composition of claim 202, wherein the barcode comprises an error-correcting code.

206. The composition of claim 202, wherein the pool of nucleic acid constructs encode a subset of possible single or multiple amino acid substitutions or deletions of a gene.

207. The composition of claim 202, wherein the pool of nucleic acid constructs comprise interference RNA coding sequences.

208. The composition of claim 202, wherein the barcode sequence comprises at least 10 unique sequences.

209. The composition of claim 202, further comprising the population of cells.

210. The composition of claim 202, wherein the genotype is based on the sequence of the distinct variant.

211. The composition of claim 202, wherein the phenotype is determined following expression of the nucleic acid construct in a population of cells.

212. The composition of claim 211, wherein the phenotype is determined by imaging the barcode in a series of sequential hybridizations and imaging with a plurality of readout probes comprising a fluorescent label, wherein the readout probes are configured to hybridize to one of the read sequences in each of the pair of read sequences of the expressed barcode sequence until all N positions in the binary code have been imaged to determine the expressed phenotype.

213. A population of cells comprising:
one or more nucleic acid constructs, wherein each construct comprises a sequence encoding a distinct variant operably linked to a barcode sequence encoding an N-bit binary barcode assigned to the distinct variant, wherein the barcode sequence comprises a pair of read sequences for each N position of the binary code wherein one of the read sequences of the pair is assigned to encode a value of “0” and the other read sequence of the pair is assigned to encode a value of “1”.

214. The population of cells of claim 213, wherein the nucleic acid constructs are introduced into the genome of the cell.

215. The population of cells of claim 213, wherein the cells are bacterial cells, mammalian cells or yeast cells.

216. The population of cells of claim 213, wherein the nucleic acid construct encodes at least two distinct variants.

217. The population of cell of claim 213, wherein the barcode sequence encodes at least a 3-bit binary barcode and wherein each possible combination of the N-bit binary barcode is present within the pool of nucleic acid constructs.

218. The population of cell of claim 213, wherein the barcode comprises an error-correcting code.

219. The population of cell of claim 213, wherein the pool of nucleic acid constructs encode a subset of possible single or multiple amino acid substitutions or deletions of a gene.

220. The population of cell of claim 213, wherein the pool of nucleic acid constructs comprise interference RNA coding sequences.

221. The population of cell of claim 213, wherein the barcode sequence comprises at least 10 unique sequences.
